(19) United States
(12) Patent Application Publication
(10) Pub. No.: US 2018/0028477 A1
(43) Pub. Date: Feb. 1, 2018

(54) TREATMENT OF CARDIAC REMODELING AND OTHER HEART CONDITIONS

(71) Applicants: C & C BIOPHARMA, LLC, Los Angeles, CA (US); UNIVERSITY OF MIAMI, Miami, FL (US)

(72) Inventors: Lin CHEN, Los Angeles, CA (US); Yongqing WU, Los Angeles, CA (US); Jianqin WEI, Miami, FL (US); Nanette BISHOPRIC, Miami, FL (US)

(21) Appl. No.: 15/724,117

(22) Filed: Oct. 3, 2017

Related U.S. Application Data
(63) Continuation of application No. 15/034,838, filed on May 5, 2016, now abandoned.
(60) Provisional application No. 61/900,007, filed on Nov. 5, 2013.

(51) Int. Cl. A61K 31/167

(52) U.S. Cl. CPC A61K 31/167

(57) ABSTRACT

The present disclosure provides novel methods for treating cardiac remodeling and heart conditions using specific compounds, as well as compositions, and pharmaceutical formulations. In some embodiments, the compounds may inhibit acetylation of MEF2 transcription factors.

### PRIORITY CLAIM

This application is a continuation of U.S. patent application Ser. No. 15/034,838, filed May 5, 2016, which is a U.S. National Phase Application of International Application No. PCT/US14/64188, filed Nov. 5, 2014, which claims priority to U.S. Provisional Patent Application No. 61/900,007, filed Nov. 5, 2013, which are incorporated herein by reference in their entirety, as if fully set forth herein.

### BACKGROUND

The heart initially responds to cardiac injury or pathological stresses by initiating cardiac remodeling. Such cardiac remodeling changes counteract different cardiac stress situations, but over the long run result in cardiac dysfunction and ultimately heart failure. Cardiac remodeling is the culmination of a complex series of transcriptional, signaling, structural, and functional events occurring within the cardiac myocyte. Cardiac remodeling also involves other cellular elements within the ventricle, including fibroblasts, the coronary vasculature, and infiltrating inflammatory cells (Bisping, 2014). Cardiac remodeling encompasses cellular changes including myocyte hypertrophy, necrosis, apoptosis, fibrosis, increased fibrillar collagen, and fibroblast proliferation.

Currently, five million Americans suffer from chronic heart failure, the final common pathway of many forms of heart dysfunctions. It is predicted that as our population ages, the direct medical costs of treatment of all forms of heart disease will triple from $272 billion in 2010 to $818 billion in 2030 (Heidenreich, 2011). Approximately 50% of heart failure diagnoses involve cardiac remodeling and associated contractile dysfunction in the absence of ischemic heart disease. Currently, there is no specific therapy for this form of heart failure, which is rapidly increasing in prevalence with aging of the population.

Although the efficacy of many therapies aimed solely at correcting a low cardiac output or reduced blood flow, including angiotensin converting enzyme (ACE) inhibitors, angiotensin receptor blockers (ARB), aldosterone antagonists, and β-adrenergic receptor blockers (β-blockers), offer symptomatic relief, they do not necessarily slow heart failure progression or reduce mortality (Cohn, 2000). To stem this enormous burden on individuals and society, there is a need in the art for treatments that target cardiac remodeling.

Certain histone deacetylase (HDAC) inhibitors demonstrate potential to reduce cardiac remodeling. These potent pan-HDAC inhibitors, including Trichostatin A (TSA), Scriptaid, and SAHA, inhibit HDACs in the low nanomolar range. However, the therapeutic benefit of HDAC inhibitors must be carefully weighed against their potential for causing toxicity. Beyond nausea and fatigue, hematologic toxicity and QT prolongation have been reported with HDAC inhibitor treatment (McKinsey, 2011). Pan-HDAC inhibition can produce transient thrombocytopenia and in some instances, myelosuppression. QT prolongation has been reported as a dose-limiting toxicity in trials with pan-HDAC inhibitors. Therefore, there is a need in the art for new compounds that can suppress cardiac remodeling without toxic side-effects.

### SUMMARY

One aspect of the invention relates to one or more compounds that can be used in the methods disclosed herein. In one embodiment, the one or more compounds may comprise a structure of Formula I:

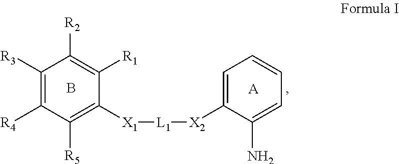

including pharmaceutically acceptable solvates, pharmaceutically acceptable prodrugs, pharmaceutically acceptable salts and pharmaceutically acceptable stereoisomers thereof, and further including mixtures thereof in all ratios, wherein:
A and B rings are independently selected from the group consisting of phenyl and pyridyl rings;
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

In another embodiment, the one or more compounds may comprise a structure of Formula II:

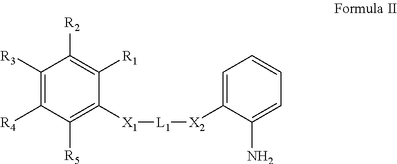

including pharmaceutically acceptable solvates, pharmaceutically acceptable prodrugs, pharmaceutically acceptable salts and pharmaceutically acceptable stereoisomers thereof, and further including mixtures thereof in all ratios, wherein:
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

In another embodiment, the one or more compounds may comprise a structure of 7MI or 8MI:

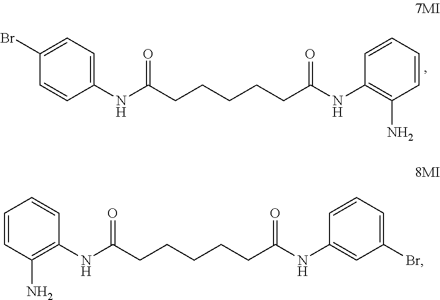 [SMILES: Nc1ccccc1NC(=O)CCCCCC(=O)Nc1ccc(Br)cc1 ; Nc1ccccc1NC(=O)CCCCCC(=O)Nc1cccc(Br)c1]

including pharmaceutically acceptable solvates, pharmaceutically acceptable prodrugs, pharmaceutically acceptable salts and pharmaceutically acceptable stereoisomers thereof, and further including mixtures thereof in all ratios.

Another aspect of the invention relates to a method of improving cardiac function in a subject comprising administering to the subject a therapeutically effective amount of one or more compounds that are disclosed herein. Another embodiment relates to the use of one or more compounds disclosed herein, or a composition or pharmaceutical formulation thereof, in the manufacture of a medicament for improving cardiac function in a subject.

Another aspect of the invention relates to a method of treating cardiac remodeling in a subject comprising administering to the subject a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating cardiac remodeling in a subject.

In some embodiments, the cardiac remodeling may manifest as symptoms including diminished cardiac contractility, increased thickness of the posterior wall of the heart, and/or increased ventricular mass. In some embodiments, the cardiac remodeling may manifest as cardiac fibrosis, myocyte hypertrophy, myocyte necrosis, myocyte apoptosis, increased fibroblast proliferation, and/or increased fibrillar collagen.

In some embodiment, the cardiac remodeling may manifest as one or more symptoms independently selected from the group consisting of: diminished diastolic function of the left ventricle, diminished systolic function of the left ventricle, diminished cardiac contractility, diminished stroke volume, diminished fractional shortening, diminished ejection fraction, increased left ventricular (LV) diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

Another aspect of the invention relates to a method of treating cardiac fibrosis in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating cardiac fibrosis in a subject.

Another aspect of the invention relates to a method of treating left ventricular dysfunction in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating left ventricular dysfunction in a subject.

In some embodiments, the left ventricular dysfunction may manifest as one or more symptoms independently selected from the group consisting of: diminished diastolic function of the left ventricle, diminished systolic function of the left ventricle, diminished stroke volume, diminished fractional shortening, diminished ejection fraction, increased LV diastolic diameter, increased LV systolic diameter, increased LV end diastolic pressure, increased LV systolic volume, increased LV diastolic volume, and/or increased LV mass.

Another aspect of the invention relates to a method of inhibiting myocyte apoptosis in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting myocyte apoptosis in a subject.

Another aspect of the invention relates to a method of inhibiting MEF2 acetylation in a subject manifesting symptoms of cardiac remodeling comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject manifesting symptoms of cardiac remodeling.

In some embodiments, the symptoms may be one or more symptoms independently selected from the group comprising: diminished diastolic function of the left ventricle, diminished systolic function of the left ventricle, diminished cardiac contractility, diminished stroke volume, diminished fractional shortening, diminished ejection fraction, increased LV diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

Another aspect of the invention relates to a method of inhibiting MEF2 acetylation in a subject having left ventricular dysfunction comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject having left ventricular dysfunction.

Another aspect of the invention relates to a method inhibiting MEF2 acetylation in a subject having cardiac fibrosis comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject having cardiac fibrosis.

In some embodiments relating to all of the methods discussed herein, the subject may have one or more symptoms independently selected from the group consisting of diminished diastolic function of the left ventricle, diminished systolic function of the left ventricle, diminished cardiac contractility, diminished stroke volume, diminished fractional shortening, diminished ejection fraction, increased LV diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

In some embodiments relating to all of the methods discussed herein, the subject may have been diagnosed with one or more conditions independently selected from the group consisting of cardiac fibrosis, hypertension, aortic stenosis, myocardial infarction, myocarditis, cardiomyopathy, valvular regurgitation, valvular disease, left ventricular dysfunction, cardiac ischemia, diastolic dysfunction, chronic angina, tachycardia, and bradycardia.

In some embodiments relating to all of the methods disclosed here, the one or more compounds may inhibit the expression of B-type natriuretic peptide (BNP) in myocytes. In some embodiments relating to all of the methods disclosed here, the one or more compounds may inhibit the expression of atrial natriuretic peptide (ANP) in myocytes. In some embodiments relating to all of the methods disclosed here, the one or more compounds may inhibit the expression of alpha-myosin heavy chain (α-MHC) in myocytes. In some embodiments relating to all of the methods disclosed here, the one or more compounds may inhibit the expression of beta-myosin heavy chain (β-MHC) in myocytes. In some embodiments relating to all of the methods disclosed here, the one or more compounds may inhibit the expression of sarco(endo)plasmic reticulum Ca2+-ATPase (SERCA) in myocytes. In some embodiments relating to all of the methods disclosed here, the one or more compounds may inhibit the expression of Collagen Type I (Col 1) or Collagen Type 3 (Col 3) in myocytes.

In some embodiments relating to all of the methods discussed herein, the one or more compounds may inhibit MEF2 acetylation. In some embodiments relating to all of the methods discussed herein, the one or more compounds may cause class IIa HDACs to re-localize from the nucleus into the cytoplasm. In some embodiments relating to all of the methods discussed herein, the one or more compounds may inhibit the binding of MEF2 to its co-factors (i.e., class IIa HDACs).

In some embodiments relating to all of the methods discussed herein, the one or more compounds may have an IC₅₀ greater than 50 μM for HDAC6 inhibition. In some embodiments relating to all of the methods discussed herein, the one or more compounds may preferentially or selectively inhibit HDAC3 over HDAC1. In some embodiments relating to all of the methods discussed herein, the one or more compounds may have an IC₅₀ greater than 1 μM for HDAC inhibition determined in an assay that detects inhibition of total histone deacetylation in a HeLa cell nuclear extract. In some embodiments relating to all of the methods discussed herein, the one or more compounds may have an IC₅₀ greater than 0.5 μM for HDAC inhibition determined in an assay that detects inhibition of total histone deacetylation in a HeLa cell nuclear extract.

### BRIEF DESCRIPTION OF THE DRAWINGS

FIG. 1 illustrates that acetylation-defective MEF2D mutants decreased myocyte hypertrophic response and acted as dominant negative inhibitors of hypertrophy. Neonatal Rat Ventricular Myocytes (NRVMs) were treated with either a vehicle (dark gray bars) or 2 μM norephinephrine (NE) (light gray bars). Hypertrophy was induced in NRVM cultures by NE in the presence of wild type (WT) or one of 2 different acetylation-defective MEF2 mutants (Mut1, MEF2D K424R or Mut2, MEF2D I423A).

FIGS. 2A-2B illustrate that MEF2 inhibitors inhibited serum-induced hypertrophy. FIG. 2A shows the chemical structures of MEF2 inhibitors (7MI and 8MI) and a control inhibitor (Trichostatin A) used in an in vitro assay with NRVMs. FIG. 2B shows that NRVMs receiving MEF2 inhibitors (7MI (black bars), 8MI (white bar), or TSA (striped bars)) displayed depressed growth response to Fetal Bovine Serum (FBS). The cells were also treated with DMSO (light gray bars) or a vehicle (dark gray bars) as a negative control. The order of potency for inhibitors was DMSO<7MI<TSA<8MI. Viability was unchanged (not shown).

FIGS. 3A-3B show that a MEF2 inhibitor blocked pressure overload-induced cardiac hypertrophy produced by transverse aortic coarctation (TAC). Mice were treated with daily injections of MEF2 inhibitor 8MI at the indicated concentrations for two weeks beginning immediately after transverse aortic banding. N=at least 3 per condition. Mice were subjected to either a sham operation (dark gray bars) or TAC (light gray bars). FIG. 3A shows the results of heart weight to tibia length ratio measured at sacrifice. FIG. 3B shows the Left Ventricular Ejection Fraction (LVEF) results. Ejection fraction, a measure of contractile function, was determined by echocardiography on a Vevo 770 ultrasound system prior to sacrifice at two weeks.

FIGS. 4A-4B illustrate that MEF2 acetylation was increased in human hearts manifesting cardiac remodeling. FIG. 4A shows representative blots of the acetylation state of MEF2 that was determined in a series of human left ventricular myocardial samples, representing 3 controls hearts (Control) and 9 cardiomyopathic hearts (Cardiac Remodeling). FIG. 4B shows a graph with the data from the immunoblots in FIG. 4A quantitated as densitometry units normalized to acetyl-lysine (n.d.u., normalized densitometry units).

FIGS. 5A-5E show the normalization of cardiac mass following TAC in 8MI-treated mice. The heart weight to tibia length ratio (HW/TL) was determined in mice 21 days after TAC or a sham operation and receiving 8MI at the indicated doses (n=4-5 per group). FIG. 5B illustrates the normalization of cardiac geometry after TAC in 8MI-treated mice with representative Masson's Trichrome-stained four-chamber cross-sections of hearts from mice treated as in FIG. 5A. Original magnification=1×. FIG. 5C shows the normalization of echocardiographic posterior wall thickness in mice treated as in FIG. 5A with 8MI at the indicated doses. FIG. 5D shows normalization of myocyte size in vivo with representative wheat germ agglutinin (WGA)-stained sections of myocardium from mice treated as in FIG. 5A and as indicated. FIG. 5E shows quantification of cell size in WGA-stained sections from cells in at least 4 myocardial sections from 3 mice per condition.

FIGS. 6A-6B show 8MI reduces fibrosis associated with pressure overload. FIG. 6A shows Masson's Trichrome staining of representative sections of myocardium from mice with indicated treatments. FIG. 6B shows fibrotic area quantified and expressed relative to the total tissue area. Data summarizes at least 4 sections from 3 mice per group.

FIGS. 7A-7G show 8MI blocks transcription and function changes associated with pressure overload. FIGS. 7A-7G show mRNA transcripts from the indicated genes as measured by quantitative realtime PCR in myocardial samples obtained from mice treated as in Example 4. FIG. 7A shows expression of Collagen Type 1 (Col 1). FIG. 7B shows expression of Collagen Type 3 (Col 3). FIG. 7C shows expression of atrial natriuretic peptide (ANP). FIG. 7D shows expression of B-type natriuretic peptide (BNP). FIG. 7E shows expression of sarco(endo)plasmic reticulum Ca2+-ATPase (SERCA). FIG. 7F shows expression of beta-myosin heavy chain (β-MHC). FIG. 7G shows expression of alpha-myosin heavy chain (α-MHC). N.d.u.=normalized transcript units.

FIGS. 8A-8H show the preservation of cardiac function in 8MI-treated mice after TAC. FIG. 8A shows ejection fraction (EF). FIG. 8B shows % fractional shortening. FIG. 8C shows stroke volume (ml). FIG. 8D shows left ventricular internal diameter at end diastolic (LViDd). FIG. 8E shows left ventricular internal diameter at end systole (LViDs). FIG. 8F shows left ventricular systolic volume (LV Vs). FIG. 8G shows left ventricular diastolic volume (LV Vd). FIG. 8H shows heart rate. In FIGS. 8A-8H, echocardiography was performed 21 days post TAC or sham operation in mice receiving 8MI (5, 20, and 40 mg/kg) or its vehicle.

FIG. 9 shows 8MI decreased pressure overload-associated MEF2 acetylation. Acetyl-MEF2 and acetyl-GATA4 were determined as described in Example 9 in myocardial lysates of TAC- or sham-operated mice treated with the indicated doses of 8MI, or its vehicle (0), as indicated. Treatment with 8MI does not reduce acetyl-MEF2 levels in non-stressed hearts. MEF2 acetylation is increased by TAC and reduced by 8MI. Myocardial lysates were immunoprecipitated with either anti-pan MEF2 or GATA4 antibodies, and probed with antibodies against MEF2, GATA4 or acetyl-lysine (control). The quantified data shown in FIG. 9 is from 3 mice per group and normalized to total acetyl-lysine (western blots are not shown).

FIG. 10 shows that 8MI prevented myocyte apoptosis during TAC in a dose dependent manner. FIG. 10 shows that myocyte apoptosis increases in TAC-operated mice in comparison to sham-operated mice. Treatment of mice with 8MI reduces myocyte apoptosis levels.

FIG. 11 shows 7MI and 8MI are metabolized by the liver better than BML-210 or TSA.

### DETAILED DESCRIPTION

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure.

Cardiac remodeling may be manifested clinically as changes in size, shape and function of the heart after cardiac injury or stress. Measures to assess left ventricular remodeling include heart size, shape, and mass, ejection fraction, end-diastolic and end-systolic volumes and peak force contraction. Cardiac remodeling may be described as a physiologic condition that may occur after myocardial infraction, cardiac ischemia, pressure overload (aortic stenosis, hypertension), inflammatory heart muscle disease (myocarditis), idiopathic dilated cardiomyopathy or volume overload (valvular regurgitation). The response of the heart to sustained load increases, as in hypertension and aortic stenosis, results in an increase in muscle mass in the overloaded chamber.

As a result of injury or stress, myocyte numbers decrease and surviving myocytes become elongated or hypertrophied as part of an initial compensatory process to maintain stroke volume after the loss of contractile tissue. The thickness of the ventricular wall also increases. Fibroblasts contribute to remodeling when activated by stress or injury by increasing collagen synthesis, thereby causing fibrosis of the ventricle.

A common scenario for remodeling is after myocardial infarction or acute ischemia. There is myocardial necrosis (cell death) and disproportionate thinning of the heart. This thin, weakened area is unable to withstand the pressure and volume load on the heart in the same manner as the other healthy tissue. As a result there is dilatation of the chamber arising from the infarct region. The initial remodeling phase after a myocardial infarction results in repair of the necrotic area and myocardial scarring that may, to some extent, be considered beneficial since there is an improvement in or maintenance of left ventricle function and cardiac output. Over time, as the heart undergoes ongoing remodeling, it becomes less elliptical and more spherical. Ventricular mass and volume increase, which together adversely affect cardiac function. Eventually, diastolic function, or the heart's ability to relax between contractions may become impaired, further causing decline.

Previous reports summarized in McKinsey showed that the transcriptional activity of MEF2 is upregulated in response to pathological stress in the heart, which in turn induces cardiac remodeling (McKinsey, 2011). Ectopic overexpression of constitutively active forms of MEF2 in the mouse heart caused dilated cardiomyopathy (McKinsey, 2011). Ectopic overexpression of either HDAC4 or HDAC9 in cultured rat cardiomyoctyes coordinately suppressed MEF2-dependent transcription and agonist-dependent cardiac hypertrophy. In contrast, disruption of the gene encoding HDAC9 in mice leads to super activation of cardiac MEF2 activity, and mouse knockouts for the HDAC5 or HDAC9 gene develop exaggerated cardiac hypertrophy in response to pressure overload and spontaneous, pathologic hypertrophy with advancing age. These previous reports summarized in McKinsey showed that class IIa HDACs are endogenous inhibitors of cardiac hypertrophy by binding MEF2 and repressing MEF2-dependent transcription.

Given this background knowledge, the compounds disclosed herein were not expected to reduce or inhibit cardiac remodeling because Jayathiliaka previously reported that the compounds disclosed herein disrupted MEF2 binding to class IIa HDACs (Jayathiliaka, 2012). Jayathiliaka previously reported that the compounds caused class IIa HDACs to re-localize from the cell nucleus into the cytoplasm. In the cytoplasm, class IIa HDAC could no longer suppress MEF2 located in the nucleus of the cell. If the compounds disrupt MEF2 binding to class IIa HDACs, MEF2 dependent transcription will no longer be repressed by the class IIa HDACs, but instead will be up-regulated. Up-regulated MEF2 dependent transcription induces cardiac remodeling. Therefore, the compounds disclosed herein were predicted to induce or increase cardiac remodeling rather than inhibit cardiac remodeling because they disrupted class IIa HDAC repression of MEF2 dependent transcription.

Certain embodiments herein relate to the unexpected results provided herein in the examples below which show how the compounds disclosed herein significantly reduced the effects of cardiac remodeling. Surprisingly, the halogen substituent of the compounds disclosed herein caused a significant reduction in myocyte cell size in serum-stimulated Neonatal Rat Ventricular Myocytes in comparison to another BML-210-like or PAOA-like compound (data not disclosed).

I. Compounds

In one embodiment, the one or more compounds that can be used in the methods disclosed herein may comprise a structure of 7MI or 8MI:

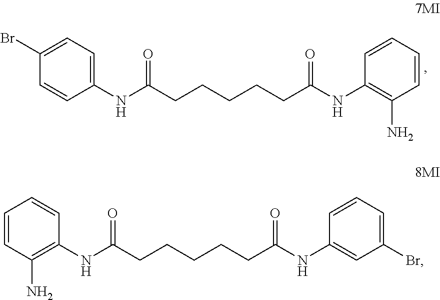 [SMILES: Nc1ccccc1NC(=O)CCCCCC(=O)Nc1ccc(Br)cc1 ; Nc1ccccc1NC(=O)CCCCCC(=O)Nc1cccc(Br)c1]

including pharmaceutically acceptable solvates, pharmaceutically acceptable prodrugs, pharmaceutically acceptable salts and pharmaceutically acceptable stereoisomers thereof, and further including mixtures thereof in all ratios.

In another embodiment, the one or more compounds disclosed herein may comprise a structure of Formula I:

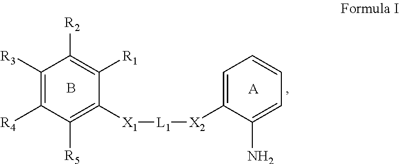

including pharmaceutically acceptable solvates, pharmaceutically acceptable prodrugs, pharmaceutically acceptable salts and pharmaceutically acceptable stereoisomers thereof, and further including mixtures thereof in all ratios, wherein:
A and B rings are independently selected from the group consisting of phenyl and pyridyl rings;
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

In some embodiments, —X1-L1-X2-is —NHC(═O)-L₁-C(═O)NH—.

In some embodiments, —X₁-L₁-X₂-is —C(═O)—NH-L₁-C(═O)NH—.

In some embodiments, at least one of R₃, R₄ and R₅ is halogen (e.g. Cl, Br, and F).

In some embodiments, A is a phenyl ring and B is a pyridyl ring.

In some embodiments, A is a phenyl ring, B is a pyridyl ring, R₁-R₃ and R₅ are hydrogen, and R₄ is a halogen (e.g. Cl, Br, and F).

In some embodiments, A is a phenyl ring, B is a pyridyl ring, R₁, R₂, R₄, and R₅ are hydrogen, and R₃ is a halogen (e.g. Cl, Br, and F).

In another embodiment, the one or more compounds disclosed herein may comprise a structure of Formula II:

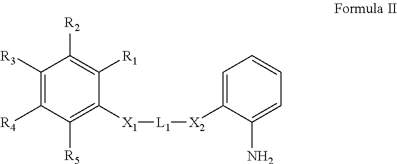

including pharmaceutically acceptable solvates, pharmaceutically acceptable prodrugs, pharmaceutically acceptable salts and pharmaceutically acceptable stereoisomers thereof, and further including mixtures thereof in all ratios, wherein R₁-R₅, X₁, X₂, and L₁ are defined the same as above.

In some embodiments, at least one of R₃, R₄ and R₄ is halogen (e.g. Cl, Br, and F).

In some embodiments, —X₁-L₁-X₂-is —NHC(═O)-L₁-C(═O)NH—.

In some embodiments, —X₁-L₁-X₂-is —C(═O)—NH-L₁-C(═O)NH—.

Unless otherwise specified, all substituents intend to include optionally substituted substituents, i.e. further substituted or not.

In some embodiments, the one or more compounds disclosed herein may inhibit MEF2 acetylation.

In some embodiments, the one or more compounds disclosed herein may inhibit the function of class IIa HDACs.

In some embodiments, the one or more compounds disclosed herein may cause class IIa HDACs to re-localize from a cell's nucleus to the cytoplasm.

In some embodiments, the one or more compounds disclosed herein may have an IC₅₀ greater than 50 μM for HDAC6 inhibition.

In some embodiments, the one or more compounds may preferentially or selectively inhibit HDAC3 over HDAC1.

In some embodiments, the one or more compounds may have an IC₅₀ greater than 0.5 μM in an assay that detects inhibition of total histone deacetylation in a HeLa cell nuclear extract.

Surprisingly, the compounds disclosed herein appear to be less toxic than pan-HDAC inhibitors. Experimental results in Examples 2 and 4 demonstrated that mice can tolerate daily doses of 8MI at 100 mg/kg for four weeks without any signs of kidney or liver disease or other adverse effects. In contrast, the tolerated daily dosage previously reported for TSA in mice was 1 mg/kg and not all of the mice survived. The weaker HDAC inhibition activity and preference or specificity for inhibiting certain HDACs likely contribute to superior toxicity profile of the compounds disclosed herein.

The compounds having structural formulas of 7MI, 8MI, Formula I and Formula II are significantly more metabolically stable in the liver than Trichostatin A (TSA) or BML-210 (see Example 11 and FIG. 11):

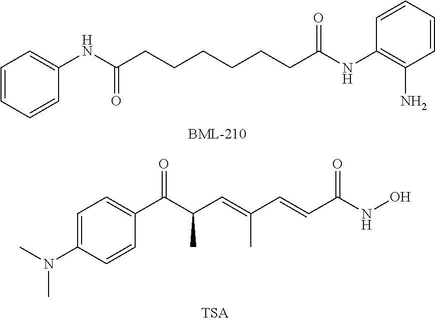 [SMILES: Nc1ccccc1NC(=O)CCCCCCC(=O)Nc1ccccc1 ; CC(/C=C/C(=O)NO)=C\[C@@H](C)C(=O)c1ccc(N(C)C)cc1 ; "TSA"]

Although the structures of BML-210 and 7MI and 8MI are related, the halogen substituent on the benzene ring surprisingly increased 7MI's and 8MI's the metabolic stability in the liver in comparison to BML-210 over time (FIG. 11). Thus, halogen substituents on pimeloyl-anilide orthoaminoanilide-like compounds unexpectedly and significantly enhance the bioavailability of the drug and reduce the likelihood of CYP-mediated drug-drug interactions. Similarly, 7MI and 8MI were also significantly more metabolically stable in the liver in comparison to TSA.

As used herein, a compound or a composition that is “pharmaceutically acceptable” is suitable for use in contact with the tissue or organ of a biological subject without excessive toxicity, irritation, allergic response, immunogenicity, or other problems or complications, commensurate with a reasonable benefit/risk ratio. If said compound or composition is to be used with other ingredients, said compound or composition is also compatible with said other ingredients.

As used herein, the term “solvate” refers to a complex of variable stoichiometry formed by a solute (e.g., compounds disclosed herein) and a solvent. Such solvents for the purpose of the invention may not interfere with the biological activity of the solute. Examples of suitable solvents include, but are not limited to, water, aqueous solution (e.g. buffer), methanol, ethanol and acetic acid. Preferably, the solvent used is a pharmaceutically acceptable solvent. Examples of suitable pharmaceutically acceptable solvents include, without limitation, water, aqueous solution (e.g. buffer), ethanol and acetic acid. Examples for suitable solvates are the mono- or dihydrates or alcoholates of the compound according to the invention.

As used herein, pharmaceutically acceptable salts of a compound refers to any pharmaceutically acceptable acid and/or base additive salt of the compound (e.g., compounds disclosed herein). Suitable acids include organic and inorganic acids. Suitable bases include organic and inorganic bases. Examples of suitable inorganic acids include, but are not limited to: hydrochloric acid, hydrofluoric acid, hydrobromic acid, hydroiodic acid, sulfuric acid and boric acid. Examples of suitable organic acids include but are not limited to: acetic acid, trifluoroacetic acid, formic acid, oxalic acid, malonic acid, succinic acid, tartaric acid, maleic acid, fumaric acid, methanesulfonic acid, trifluoromethanesulfonic acid, benzoic acid, glycolic acid, lactic acid, citric acid and mandelic acid. Examples of suitable inorganic bases include, but are not limited to: ammonia, hydroxyethylamine and hydrazine. Examples of suitable organic bases include, but are not limited to, methylamine, ethylamine, trimethylamine, triethylamine, ethylenediamine, hydroxyethylamine, morpholine, piperazine and guanidine. The invention further provides for the hydrates and polymorphs of all of the compounds described herein.

II. Compositions

The compounds disclosed herein may contain one or more chiral atoms, or may otherwise be capable of existing as two or more stereoisomers, which are usually enantiomers and/or diastereomers. Accordingly, compositions comprising the compounds disclosed herein may include mixtures of stereoisomers or mixtures of enantiomers, as well as purified stereoisomers, purified enantiomers, stereoisomerically enriched mixtures, or enantiomerically enriched mixtures. The composition provided herein also include the individual isomers of the compound represented by the structures described above as well as any wholly or partially equilibrated mixtures thereof. The compositions disclosed herein also cover the individual isomers of the compound represented by the structures described above as mixtures with isomers thereof in which one or more chiral centers are inverted. Also, it is understood that all tautomers and mixtures of tautomers of the structures described above are included within the scope of the structures and preferably the structures corresponding thereto.

Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as camphorsulfonic acid. Also advantageous is enantiomer resolution with the aid of a column filled with an optically active resolving agent. The diastereomer resolution can also be carried out by standard purification processes, such as, for example, chromatography or fractional crystallization.

It is also possible to obtain optically active compounds comprising the structure of the compounds disclosed herein by the methods described above by using starting materials which are already optically active.

III. Pharmaceutical Formulations

As used herein, a pharmaceutical formulation comprises a therapeutically effective amount of one or more of the compounds or compositions thereof disclosed herein. In certain embodiments, the pharmaceutical formulation further comprises a pharmaceutically acceptable carrier.

As used herein, a “therapeutically effective amount,” “therapeutically effective concentration” or “therapeutically effective dose” is an amount which, as compared to a corresponding subject who has not received such amount, results in improved treatment, healing, prevention, or amelioration of a disease, disorder, or side effect, or a decrease in the rate of advancement of a disease or disorder.

This amount will vary depending upon a variety of factors, including but not limited to the characteristics of the compounds, compositions, or pharmaceutical formulations thereof (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the compound, composition, or pharmaceutical formulation thereof is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more compounds, compositions, or pharmaceutical formulations thereof and adjusting the dosage accordingly. A typical dosage may range from about 0.1 mg/kg to about 100 mg/kg or more, depending on the factors mentioned above. In other embodiments, the dosage may range from about 0.1 mg/kg to about 100 mg/kg; or about 1 mg/kg to about 100 mg/kg; or about 5 mg/kg up to about 100 mg/kg. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein for additional guidance for determining a therapeutically effective amount.

As used herein, the term “about” refers to ±10%, ±5%, or ±1%, of the value following “about.”

A “pharmaceutically acceptable carrier” is a pharmaceutically-acceptable material, composition or vehicle, such as a liquid or solid filler, diluent, excipient, solvent or encapsulating material, involved in carrying or transporting an active ingredient from one location, body fluid, tissue, organ (interior or exterior), or portion of the body, to another location, body fluid, tissue, organ, or portion of the body. Each carrier is “pharmaceutically acceptable” in the sense of being compatible with the other ingredients, e.g., the compounds described herein or other ingredients, of the formulation and suitable for use in contact with the tissue or organ of a biological subject without excessive toxicity, irritation, allergic response, immunogenicity, or other problems or complications, commensurate with a reasonable benefit/risk ratio.

Pharmaceutically acceptable carriers are well known in the art and include, without limitation, (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; and (21) other non-toxic compatible substances employed in pharmaceutical formulations.

The pharmaceutical formulations disclosed herein may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of the one or more compounds disclosed herein in a pharmaceutical formulation can vary widely, and will be selected primarily based on fluid volumes, viscosities, body weight and the like in accordance with the particular mode of administration selected and the biological subject's needs. For example, the concentration of the compounds disclosed herein can be about 0.0001% to about 100%, about 0.001% to about 50%, about 0.01% to about 30%, about 0.1% to about 20%, or about 1′)/0 to about 10% wt.

A suitable pharmaceutically acceptable carrier may be selected taking into account the chosen mode of administration, and the physical and chemical properties of the compounds.

One skilled in the art will recognize that a pharmaceutical formulation containing the one or more compounds disclosed herein or compositions thereof can be administered to a subject by various routes including, without limitation, orally or parenterally, such as intravenously. The composition may also be administered through subcutaneous injection, subcutaneous embedding, intragastric, topical, and/or vaginal administration. The composition may also be administered by injection or intubation.

In one embodiment, the pharmaceutical carrier may be a liquid and the pharmaceutical formulation would be in the form of a solution. In another embodiment, the pharmaceutically acceptable carrier is a solid and the pharmaceutical formulation is in the form of a powder, tablet, pill, or capsules. In another embodiment, the pharmaceutical carrier is a gel and the pharmaceutical formulation is in the form of a suppository or cream.

A solid carrier can include one or more substances which may also act as flavoring agents, lubricants, solubilizers, suspending agents, fillers, glidants, compression aids, binders or table-disintegrating agents, it can also be an encapsulating material. In powders, the carrier is a finely divided solid that is in admixture with the finely divided active ingredient. In tablets, the active-ingredient is mixed with a carrier having the necessary compression properties in suitable proportions and compacted in the shape and size desired. The powders and tablets preferably contain up to about 99% of the one or more compounds disclosed herein. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Besides containing an effective amount of the one or more compounds described herein or compositions thereof, the pharmaceutical formulations provided herein may also include suitable diluents, preservatives, solubilizers, emulsifiers, adjuvant and/or carriers.

The pharmaceutical formulation can be administered in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

Additional pharmaceutical formulations will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/0082948 which is incorporated herein by reference as if fully set forth herein for the techniques of controlled release of porous polymeric microparticles for the delivery of pharmaceutical formulations. Additional examples of sustained-release preparations include semipermeable polymer matrices in the form of shaped articles, e.g. films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate or poly-D (−)-3-hydroxybutyric acid. Sustained-release compositions also include liposomes, which can be prepared by any of several methods known in the art.

IV. Methods of Treatment

One aspect of the invention relates to a method of treating cardiac remodeling in a subject comprising administering to the subject a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating cardiac remodeling in a subject.

In some embodiments, the cardiac remodeling may manifest as diminished cardiac contractility, increased thickness of the posterior wall of the heart, and/or increased ventricular mass. In some embodiments, the cardiac remodeling may manifest as cardiac fibrosis, myocyte hypertrophy, myocyte necrosis, myocyte apoptosis, increased fibroblast proliferation, and/or increased fibrillar collagen.

In some embodiments, the cardiac remodeling may manifest as one or more symptoms independently selected from the group consisting of: diminished diastolic function of the left ventricle, diminished systolic function of the left ventricle, diminished cardiac contractility, diminished stroke volume, diminished fractional shortening, diminished ejection fraction, increased left ventricular (LV) diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

Another aspect of the invention relates to a method of improving cardiac function in a subject comprising administering to the subject a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for improving cardiac function in a subject.

In some embodiments the cardiac function may be improved by enhancing cardiac contractility. In some embodiment the cardiac function may be improved by diminishing cardiac fibrosis in the subject. In some embodiments, the cardiac function may be improved by reducing the thickness of the posterior wall of the heart. In some embodiments, the cardiac function may be improved by decreasing the ventricular mass. In some embodiments, the cardiac function may be improved by improving the diastolic and/or systolic function of the left or right ventricle. In other embodiments, the cardiac function may be improved by increasing the stroke volume, fractional shortening, and/or ejection fraction. In certain embodiment, the cardiac function may be improved by decreasing the LV diastolic and/or systolic diameters. In some embodiments, the cardiac function may be improved by decreasing the LV end diastolic pressure. In some embodiments, the cardiac function may be improved by reducing the LV end systolic and/or end diastolic volume. In certain embodiments, the cardiac function may be improved by decreasing the ventricular wall stress and/or ventricular wall tension.

In some embodiments, the cardiac function may be improved by decreasing the circulating B-type natriuretic peptide (BNP) levels. Expression of the gene encoding B-type natriuretic peptide is enhanced in ventricular myocytes during pathological cardiac hypertrophy, and circulating BNP levels are used clinically as a surrogate measure of heart failure. In some embodiments, the cardiac function may be improved by decreasing the expression of the alpha-myosin heavy chain and/or the beta-myosin heavy chain.

Another aspect of the invention relates to a method of treating cardiac fibrosis in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating cardiac fibrosis in a subject.

Another aspect of the invention relates to a method of treating left ventricular dysfunction in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating left ventricular dysfunction in a subject.

In some embodiments, the left ventricular dysfunction may manifest as one or more symptoms independently selected from the group consisting of: diminished diastolic function of the left ventricle, diminished systolic function of the left ventricle, diminished stroke volume, diminished fractional shortening, diminished ejection fraction, increased LV diastolic diameter, increased LV systolic diameter, increased LV end diastolic pressure, increased LV systolic volume, increased LV diastolic volume, and/or increased LV mass.

Another aspect of the invention relates to a method of treating right ventricular dysfunction in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating right ventricular dysfunction in a subject.

In some embodiments, the right ventricular dysfunction may manifest as one or more symptoms independently selected from the group consisting of: diminished diastolic function of the right ventricle, diminished systolic function of the right ventricle, diminished stroke volume, diminished fractional shortening, diminished ejection fraction, increased right ventricular (RV) diastolic diameter, increased RV systolic diameter, increased RV end diastolic pressure, increased RV systolic volume, increased RV diastolic volume, and/or increased RV mass.

Another aspect of the invention relates to a method of treating cardiac hypertrophy in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for treating cardiac hypertrophy in a subject.

Another aspect of the invention relates to a method of inhibiting myocyte apoptosis in a subject comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting myocyte apoptosis in a subject.

Another aspect of the invention relates to a method of inhibiting MEF2 acetylation in a subject manifesting symptoms of cardiac remodeling comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject manifesting symptoms of cardiac remodeling.

In some embodiments, the symptoms may be one or more symptoms independently selected from the group comprising: diminished diastolic function of the left ventricle, diminished systolic function of the left ventricle, diminished cardiac contractility, diminished stroke volume, diminished fractional shortening, diminished ejection fraction, increased LV diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

Another aspect of the invention relates to a method of inhibiting MEF2 acetylation in a subject having left ventricular dysfunction comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject having left ventricular dysfunction.

Another aspect of the invention relates to a method inhibiting MEF2 acetylation in a subject having cardiac fibrosis comprising administering a therapeutically effective amount of one or more compounds, compositions, or pharmaceutical formulations disclosed herein. Another embodiment relates to the use of one or more compounds disclosed herein, or a composition or pharmaceutical formulation thereof, in the manufacture of a medicament for inhibiting MEF2 acetylation in a subject having cardiac fibrosis.

In some embodiments relating to all of the methods disclosed herein, the subject may have one or more independently selected from the group consisting of diminished diastolic function of the left ventricle, diminished systolic function of the left ventricle, diminished cardiac contractility, diminished stroke volume, diminished fractional shortening, diminished ejection fraction, increased LV diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

In some embodiments relating to all of the methods disclosed herein, the subject may have been diagnosed with one or more conditions independently selected from the group consisting of: cardiac fibrosis, hypertension, aortic stenosis, myocardial infarction, myocarditis, cardiomyopathy, valvular regurgitation, valvular disease, left ventricular dysfunction, cardiac ischemia, diastolic dysfunction, chronic angina, tachycardia, and bradycardia.

In some embodiments relating to all of the methods disclosed here, the one or more compounds may inhibit the expression of B-type natriuretic peptide (BNP) in myocytes. In some embodiments relating to all of the methods disclosed here, the one or more compounds may inhibit the expression of atrial natriuretic peptide (ANP) in myocytes. In some embodiments relating to all of the methods disclosed here, the one or more compounds may inhibit the expression of alpha-myosin heavy chain (α-MHC) in myocytes. In some embodiments relating to all of the methods disclosed here, the one or more compounds may inhibit the expression of beta-myosin heavy chain (β-MHC) in myocytes. In some embodiments relating to all of the methods disclosed here, the one or more compounds may inhibit the expression of sarco(endo)plasmic reticulum Ca2+-ATPase (SERCA) in myocytes. In some embodiments relating to all of the methods disclosed here, the one or more compounds may inhibit the expression of Collagen Type I (Col 1) or Collagen Type 3 (Col 3) in myocytes.

In some embodiments relating to all of the methods disclosed herein, the one or more compounds may inhibit MEF2 acetylation. In some embodiments relating to all of the methods discussed herein, the one or more compounds may cause class IIa HDACs to re-localize from the nucleus into the cytoplasm. In other embodiments relating to all of the methods discussed herein, the one or more compounds may inhibit the binding of MEF2 to its co-factors (i.e., class IIa HDACs).

In some embodiments relating to all of the methods disclosed herein, the one or more compounds may have an IC₅₀ greater than 50 μM for HDAC6 inhibition. In some embodiments relating to all of the methods discussed herein, the one or more compounds may preferentially or selectively inhibit HDAC3 over HDAC1. In some embodiments relating to all of the methods discussed herein, the one or more compounds may have an IC₅₀ greater than 1 μM for HDAC inhibition determined in an assay that detects inhibition of total histone deacetylation in a HeLa cell nuclear extract. In other embodiments relating to all of the methods discussed herein, the one or more compounds may have an IC₅₀ greater than 0.5 μM for HDAC inhibition determined in an assay that detects inhibition of total histone deacetylation in a HeLa cell nuclear extract.

In some embodiments relating to all of the methods disclosed herein, the administering may comprise oral administration of the one or more compounds. As illustrated in Example 11, 8MI was more stable in the liver than BML-210 or TSA, thereby increasing its bioavailability and effectiveness in a dosage administered orally.

In some embodiments relating to all of the methods disclosed herein, the administering may comprises intravenous administration.

Optimal dosages to be administered may be determined by those skilled in the art, and will vary with the particular compound, composition, or formulation being used, the strength of the preparation, the mode of administration, and the advancement of the disease condition. Additional factors depending on the particular subject being treated, include, without limitation, subject age, weight, gender, diet, time of administration, time and frequency of administration, drug combination(s), reaction sensitivities, and response to therapy. Administration of the compound, composition, or pharmaceutical formulation may be effected continuously or intermittently. In any treatment regimen, the compound, composition, or pharmaceutical formulation may be administered to a subject either singly or in a cocktail containing two or more compounds or compositions thereof, other therapeutic agents, compositions, or the like, including, but not limited to, tolerance-inducing agents, potentiators and side-effect relieving agents. All of these agents are administered in generally-accepted efficacious dose ranges such as those disclosed in the Physician's Desk Reference, 41st Ed., Publisher Edward R. Barnhart, N.J. (1987), which is herein incorporated by reference as if fully set forth herein. In certain embodiments, an appropriate dosage level will generally be about 0.001 to about 50 mg per kg subject body weight per day that can be administered in single or multiple doses. Preferably, the dosage level will be about 0.005 to about 25 mg/kg, per day; more preferably about 0.01 to about 10 mg/kg per day; and even more preferably about 0.05 to about 1 mg/kg per day. In some embodiments, the daily dosage may be between about 10⁻⁶g/kg to about 5 g/kg of body weight.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

In some embodiments, the one or more compounds disclosed herein or compositions or pharmaceutical formulations thereof may be administered in combination with one or more additional therapeutic agents in the methods provided herein. “In combination” or “in combination with,” as used herein, means in the course of treating the same cardiac hypertrophy in the same subject using two or more agents, drugs, treatment regimens, treatment modalities or a combination thereof, in any order. This includes simultaneous administration (in the same or separate formulations), as well as administration in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

Examples of therapeutic agents that may be administered in combination with the compounds disclosed herein or compositions or pharmaceutical formulations thereof include, but are not limited to, β-adrenergic receptor blocking agents, antihypertensive drugs, aryloxyalkanoic acid/fibric acid derivatives, resins/bile acid sequesterants, HMG CoA Reductase inhibitors, nicotinic acid derivatives, thyroid hormones and analogs, antihyperlipoproteinemics, antiarteriosclerotics, antithrombotic/fibrinolytic agents, anticoagulants, antiplatelet agents, thrombolytic agents, blood coagulants, anticoagulant antagonists, thrombolytic agent antagonists and antithrombotics, antiarrhythmic agents, sodium channel blockers, β blockers, repolarization prolonging agents, calcium channel blockers/antagonist, antiarrhythmic agents, a blockers, α/β blockers, anti-angiotension II agents, sympatholytics, vasodilators, vasopressors, treatment agents for congestive heart failure, afterload-preload reduction agents, diuretics, inotropic agents, and/or antianginal agents.

In another embodiment, the therapeutic agent is an anti-cancer agent. Anti-cancer agents that may be used in accordance with certain embodiments described herein are often cytotoxic or cytostatic in nature and may include, but are not limited to, alkylating agents; antimetabolites; anti-tumor antibiotics; topoisomerase inhibitors; mitotic inhibitors; hormones (e.g., corticosteroids); targeted therapeutics (e.g., selective estrogen receptor modulators (SERMs)); toxins; immune adjuvants, immunomodulators, and other immunotherapeutics (e.g., therapeutic antibodies and fragments thereof, recombinant cytokines and immunostimulatory molecules—synthetic or from whole microbes or microbial components); enzymes (e.g., enzymes to cleave prodrugs to a cytotoxic agent at the site of the tumor); nucleases; antisense oligonucleotides; nucleic acid molecules (e.g., mRNA molecules, cDNA molecules or RNAi molecules such as siRNA or shRNA); chelators; boron compounds; photoactive agents and dyes. Examples of anti-cancer agents that may be used as therapeutic agents in accordance with certain embodiments of the disclosure include, but are not limited to, 13-cis-retinoic acid, 2-chlorodeoxyadenosine, 5-azacitidine, 5-fluorouracil, 6-mercaptopurine, 6-thioguanine, actinomycin-D, adriamycin, aldesleukin, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bexarotene, bicalutamide, bortezomib, bleomycin, busulfan, calcium leucovorin, citrovorum factor, capecitabine, canertinib, carboplatin, carmustine, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, ozogamicin, goserelin, granulocyte-colony stimulating factor, granulocyte macrophage-colony stimulating factor, hexamethylmelamine, hydrocortisone hydroxyurea, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, PEG Interferon, pegaspargase, pegfilgrastim, PEG-L-asparaginase, pentostatin, plicamycin, prednisolone, prednisone, procarbazine, raloxifene, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tretinoin, trimitrexate, alrubicin, vincristine, vinblastine, vindestine, vinorelbine, vorinostat, and zoledronic acid.

Therapeutic antibodies and functional fragments thereof, that may be used as anti-cancer agents in accordance with certain embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab and other antibodies associated with specific diseases listed herein.

Toxins that may be used as anti-cancer agents in accordance with certain embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as therapeutic agents in accordance with certain embodiments of the disclosure include, but are not limited to, ³²P, ⁸⁹Sr, ⁹⁰Y, ^{99m}Tc, ⁹⁹Mo, ¹³¹I, ¹⁵³Sm, ¹⁷⁷Lu, ¹⁸⁶Re, ²¹³Bi, ²²³Ra and ²²⁵Ac.

The frequency of dosing will depend upon the pharmacokinetic parameters of the therapeutic agents in the pharmaceutical formulation (e.g. the one or more compounds disclosed herein) used. Typically, a pharmaceutical formulation is administered until a dosage is reached that achieves the desired effect. The formulation may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data. Long-acting pharmaceutical formulations may be administered every 3 to 4 days, every week, or biweekly depending on the half-life and clearance rate of the particular formulation.

Another aspect relates to the use of one or more compounds disclosed herein or compositions or pharmaceutical formulations thereof in the manufacture of a medicament for the treatment of a condition regulatable by one or more transcription factors and/or cofactors. For this aspect, the one or more compounds or compositions or pharmaceutical formulations thereof, the transcription factors and/or cofactors, and the conditions regulatable by the transcription factor and/or cofactor are the same as disclosed above, and the treatment of the condition is the same as described supra.

Unless the context clearly requires otherwise, throughout the description and the claims, the words “comprise,” “comprising,” and the like are to be construed in an inclusive sense (i.e., to say, in the sense of “including, but not limited to”), as opposed to an exclusive or exhaustive sense. The words “herein,” “above,” “below,” “supra,” and words of similar import, when used in this application, refer to this application as a whole and not to any particular portions of this application. Where the context permits, words in the above Detailed Description using the singular or plural number may also include the plural or singular number respectively. The words “or,” and “and/or” in reference to a list of two or more items, covers all of the following interpretations of the word: any of the items in the list, all of the items in the list, and any combination of the items in the list.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### 7MI and 8MI Inhibited Cardiomyocyte Hypertrophy In Vitro

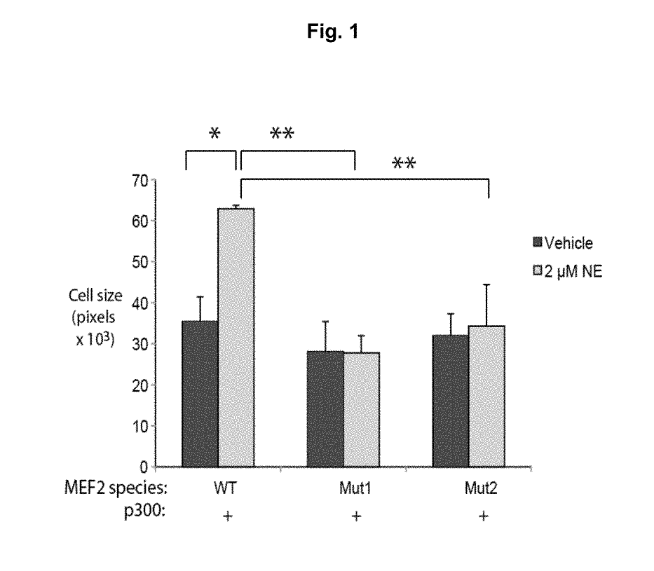

Two different MEF2 mutant constructs, Mut1 and Mut2, that lack the ability to be acetylated by p300 were used as a positive control for effective interruption of MEF2 signaling. Transfection of either of these mutants, but not of wild-type MEF2 (WT), in the presence of p300, completely blocked norepinephrine-induced cardiomyocyte hypertrophy (FIG. 1).

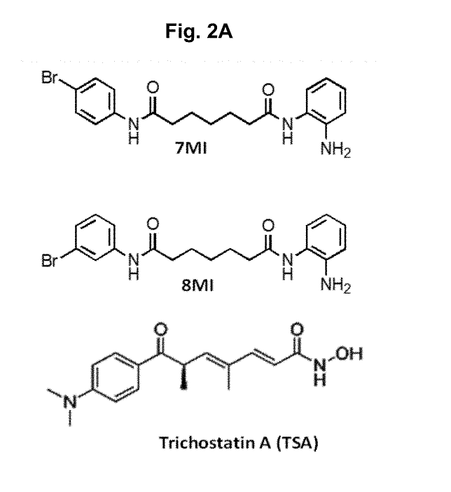

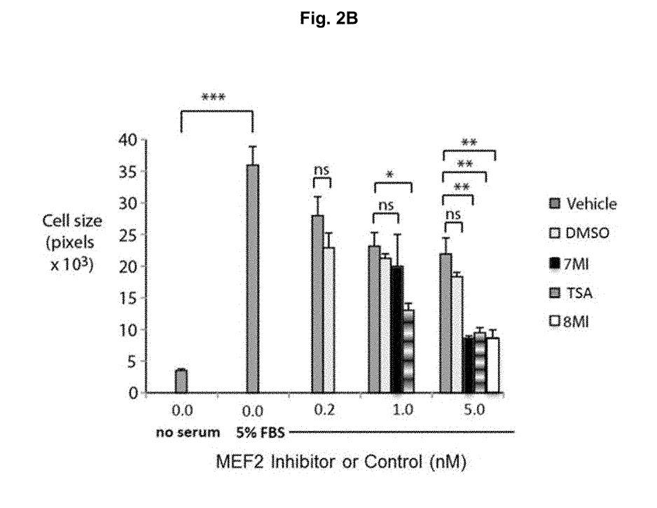

Next, the ability of 7MI and 8MI to inhibit hypertrophy in vitro was tested (FIG. 2A). A number of agonists, including al-adrenergic compounds (such as norepinephrine), angiotensin II, and growth factors including IGF-1, have previously been shown to induce hypertrophy in the in vitro system used herein. However, fetal calf serum, which contained a rich variety of growth factors, was chosen as the most robust and multifactorial stimulus for cardiomyocyte growth available, reasoning that a similarly robust and powerful inhibitor would be required to block this hypertrophy. Pre-treatment of Neonatal Rat Ventricular Myocytes (NRVMs) with a range of doses of test compounds reduced serum-stimulated myocyte growth with an order of potency similar to their previously observed in vitro transcriptional repression activity (FIG. 2B and data not shown). Taken together, these results show that inhibiting MEF2 activation in cardiac myocytes using the compounds 7MI and 8MI was sufficient to block myocyte hypertrophy in response to both narrow and broadly-acting growth signals.

### Example 2

### 7MI and 8MI Inhibited Cardiomyocyte Hypertrophy In Vivo

Based on the results in Example 1, the most potent inhibitor (8MI) was tested in an in vivo assay of pressure overload produced by transverse aortic coarctation (TAC). TAC has been used extensively to model the clinical hypertrophic stimuli of hypertension, aortic valve disease, and other types of pressure overload. In vivo pressure overload was created in wild type C57/B16 by creation of a surgical restriction in the transverse aorta between the origins of the right and left carotid arteries as previously described in Wei et. al. 2008. Control littermates were subjected to a sham operation. Surgical mortality was <5%. Trans-aortic gradients were determined by simultaneous measurements from the right and left carotid arteries using Statham pressure transducers (model P23XL, Viggo-Spectramed, Oxnard, Calif.) zeroed at the level of the right atrium. Pressures were continuously recorded as described in Wei et. al. 2008. Paired TAC and control animals were sacrificed at defined intervals after surgery and hearts were removed for analysis.

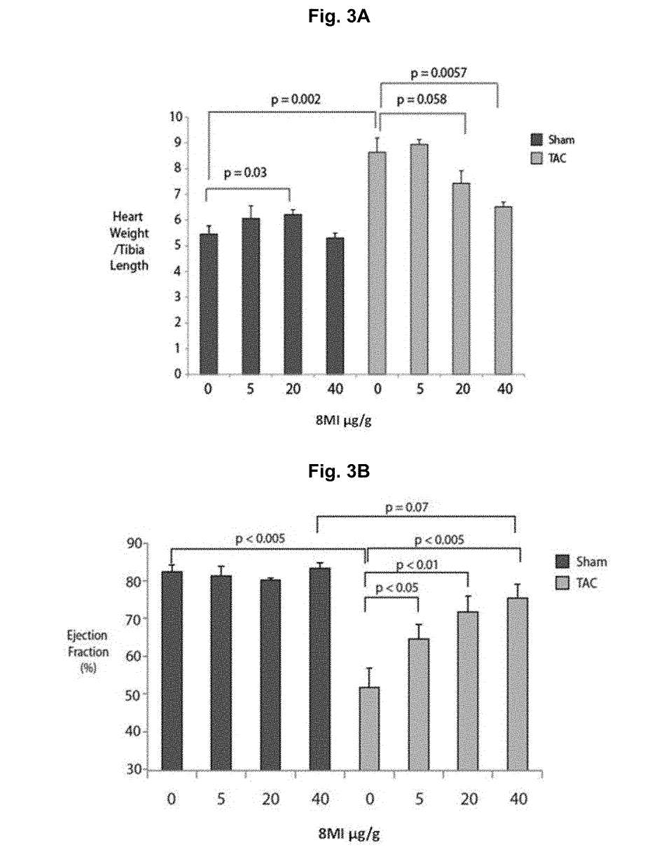

C57/B16 mice were treated with 8MI for two weeks and evaluated for cardiac function by echocardiography and for cardiac mass using a standard index of heart weight to tibia length (HW/TL) after sacrifice. TAC caused a significant >50% increase in cardiac mass two weeks after surgery (FIG. 3A, 0 μg/g of 8MI, light gray bar). Treatment with 8MI significantly blunted this increase, in a dose-dependent manner (FIG. 3A, 20 μg/g and 40 μg/g of 8MI, light gray bars). TAC-associated hypertrophy was accompanied by the development of systolic heart failure as reflected in a reduction from ˜80% to ˜50% in Left Ventricular Ejection Fraction (LVEF) (FIG. 3B, compare 0 μg/g of 8MI, dark gray bar (˜80%) with 0 μg/g of 8MI, light gray bar (˜50%)). Unexpectedly, treatment with 8MI largely prevented this impairment of function, with beneficial effects seen even at the lowest dose (FIG. 3B, 5 μg/g, 20 μg/g, and 40 μg/g of 8MI, light gray bars). 8MI showed no apparent toxicity up to 20 μM in the culture of a variety of cells. Mice treated with 8MI 100 mg/Kg daily for four weeks showed no sign of kidney or liver damage or other adverse effects, suggesting that 8MI was well tolerated in the animal model. Additionally, no mortality was seen in either group.

These findings demonstrate the benefits of targeting MEF2 activation to reduce the hypertrophic response to hemodynamic stress. Further, these results show that blunting hypertrophy by this mechanism did not impair systolic function during adaptation to a pressure load. In fact, the opposite was shown to be the case: blocking hypertrophic growth was associated with improved function, providing a strong rationale for targeting hypertrophy to delay or prevent heart failure.

### Example 3

### MEF2 Acetylation is Increased in Human Hearts Undergoing Cardiac Remodeling

The acetylation state of MEF2 was determined in a series of human left ventricular myocardial samples, representing 3 hearts with no symptoms of cardiac remodeling or heart failure (Control) and 9 hearts showing symptoms of cardiac remodeling and of heart failure. Table 1 provides characteristics for the controls hearts and the 9 symptomatic hearts.

TABLE 1
Characteristics of human subjects analyzed

| | | Heart | | | |
|---|---|---|---|---|---|
| | | Weight | | | |
| Gender | Condition | (gms) | Age | Ethnicity | Notes |
| Male | Control | 414 | 55 | white | Normal |
| | | | | | anatomy |
| Male | Control | Not | 46 | Black | Stent, apical |
| | | determined | | | scarring |
| Male | Control | 472 | 40 | White | Triple vessel |
| | | | | | coronary artery |
| | | | | | disease, septal |
| | | | | | infarct |
| Male | Cardiac | 476.6 | 40 | White | Severe |
| | Remodeling | | | | concentric |
| | | | | | hypertrophy |
| | | | | | and |
| | | | | | chamber |
| | | | | | dilatation |
| Male | Cardiac | 518 | 42 | Black | Biventricular |
| | Remodeling | | | | dilatation, |
| | | | | | hypertrophy, |
| | | | | | and interstitial |
| | | | | | fibrosis |
| Male | Cardiac | 840 | 51 | Unknown | Hypertrophic |
| | Remodeling | | | | |
| Female | Cardiac | 280 | 33 | white | Hypertrophic, |
| | Remodeling | | | | aortic value |
| | | | | | prosthesis |
| Female | Cardiac | unknown | 59 | white | Posterior |
| | Remodeling | | | | infarction, |
| | | | | | fibrosis |
| Male | Cardiac | 548 | 67 | white | Concentric |
| | Remodeling | | | | hypertrophy, |
| | | | | | and patchy |
| | | | | | fibrosis |
| Male | Cardiac | 360 | 39 | white | Concentric |
| | Remodeling | | | | hypertrophy |
| Female | Cardiac | 526 | 62 | black | Hypertrophy |
| | Remodeling | | | | |
| Male | Cardiac | 547 | 62 | while | Ischemic |
| | Remodeling | | | | cardio- |
| | | | | | myopathy, |
| | | | | | hypertrophy |

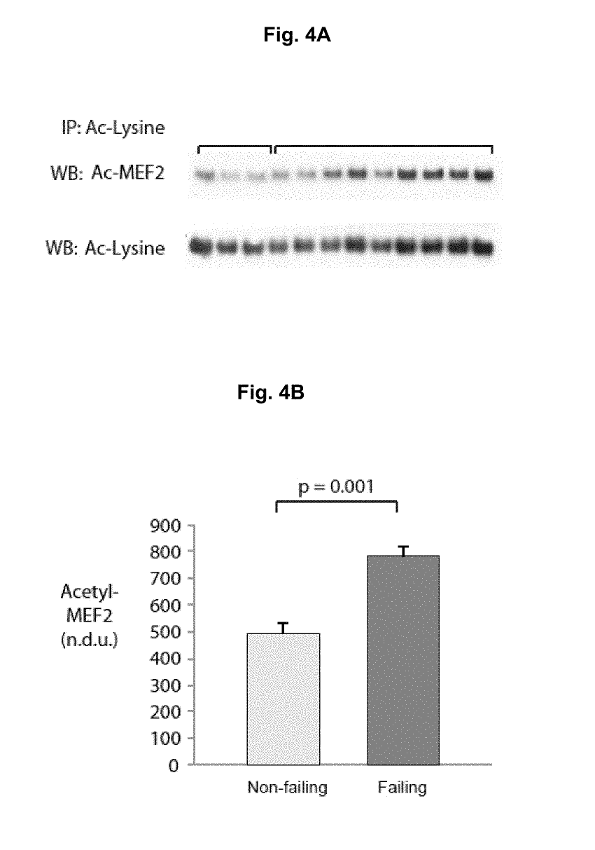

The human left ventricular myocardial samples were obtained from anonymous donors through the Cooperative Human Tissue Network and maintained at ˜80° C. until used. The tissue was harvested within 4 hours post-demise. The samples were homogenized, and the subsequent lysates were immunoprecipitated with an anti-acetyl-lysine antibody (Upstate, Charlottesville, Va., USA). Immunoprecipitates were electrophoretically separated and immunoblotted anti-MEF2 antibodies (Santa Cruz Biotechnology, Santa Cruz, Calif., USA) and anti-acetyl-lysine as a loading control. Representative blots are shown in FIG. 4A. The graph in FIG. 4B quantitates the data from the immunoblots as densitometry units normalized to Acetyl-Lys (n.d.u., normalized densitometry units). Acetylation of MEF2 species was elevated in heart samples show symptoms of cardiac remodeling relative to the Control heart samples (FIG. 4B). Results show that MEF2 acetylation is increased in heart conditions undergoing cardiac remodeling.

### Example 4

### 8MI Prevented Cardiac Remodeling In Vivo

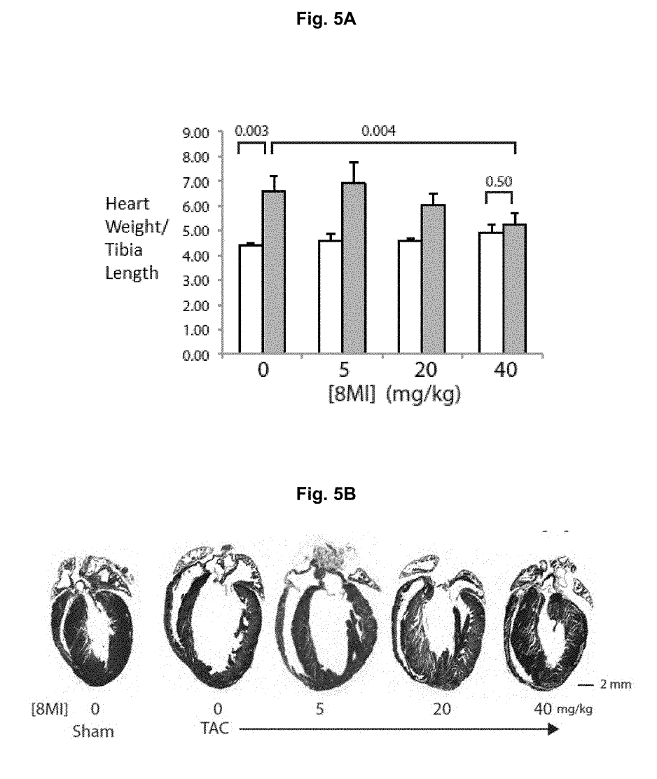

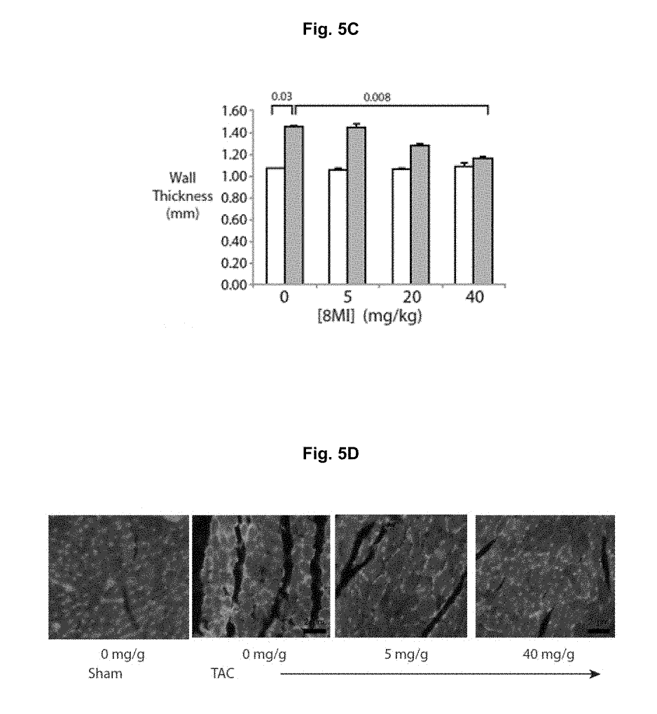

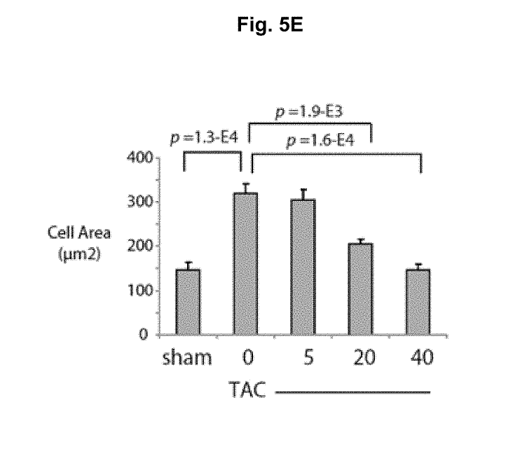

The effects of 8MI on hypertrophy in a model of moderate pressure overload were tested further (FIGS. 5A-5E). 8MI or its vehicle (DMSO) was administered over a range of concentrations immediately prior to and for 21 days following transverse aortic coarctation (TAC). All experiments were performed on 2-3 month old wild type C57/BL/6 mice. Cardiac hypertrophy was induced by TAC as described in Wei et. al. 2008. Pressure gradients induced by TAC were evaluated postoperatively by pulsed-wave Doppler echocardiography to confirm equivalent gradients in all animals (45±5 mmHg). 8MI was delivered via tail vein injection one day prior to surgical intervention and then daily for 21 days. Blood samples were obtained at sacrifice on day 21 after surgery, and serum was frozen at 80° C. until analysis

Masson's Trichrome was used to visualize cardiac four-chamber anatomy. Paraffin embedded sections were used for staining (FIG. 5B). Representative wheat germ agglutinin (WGA)-stained sections of myocardium from the mice were prepared. For echocardiography (FIG. 5C), mice were placed under anesthesia with 40 mg/kg ketamine and 5 mg/kg xylocaine and secured in a supine position. Mice were evaluated using 40-hertz transducer on a Visual Sonics 770 High Resolution Imaging System. B-mode in the short and long axis view of the ventricle was used to evaluate wall motion defects of ventricle and M-mode in long axis view used for the interventricular septal thickness, posterior wall thickness and the left ventricular dimensions in systole and diastole.

Hematoxylin and Eosin (HE) and FITC conjugated WGA (from Invitrogen) were used to evaluate myocardial cell size (FIGS. 5D and 5E). The sizes of cells from at least 4 myocardial sections from 3 mice per condition were measured (FIG. 5E). WGA samples were counterstained with DAPI and images were merged from the DAPI and FITC channels.

At the end of 21 days, in vehicle-treated animals, TAC induced a 50% increase in normalized heart weight (heart weight/tibia length, HW/TL) compared with mice undergoing a sham operation (FIG. 5A). In FIG. 5A, the white bars represent mice that had the sham operation, and the black bars represent the mice that had the TAC-operation. Administration of 8MI blunted the increase of heart weight in a dose-dependent manner, essentially reducing HW/TL at the highest dose used (40 mg/kg) to normal levels as indicated by the white bars (FIG. 5A). In comparison to previously published results for TSA, 8MI showed a significantly superior effect.

Four-chamber sections of the hearts demonstrated that treatment with 8MI also prevented remodeling of the myocardium, again in a dose-dependent manner (FIG. 5B). Confirming these findings, echocardiographic wall thickness was increased by 35.9%±1.0% in vehicle-treated animals, but only 6.9%±1.4% in mice at the highest dose of 8MI (FIG. 5C). Myocyte cross-sectional area increased by 2.2-fold in response to TAC (TAC, 319±22 μm^{2 vs.} sham, 146±17); treatment with 8MI effectively eliminated this 2.2-fold increase (FIGS. 5D and 5E). Again, in comparison to previously published results for TSA, 8MI showed a significantly superior effect in reducing myocyte cross sectional area.

### Example 5

### 8MI Inhibited Ventricular Fibrosis In Vivo

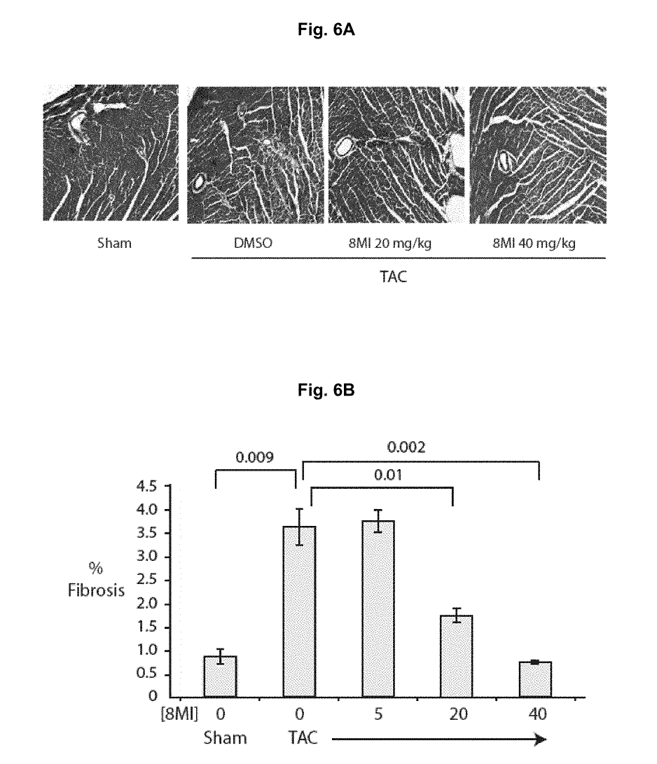

Using cardiac tissue samples obtained from the same mice treated under the experimental conditions described in Example 4, Masson's Trichrome was used to stain fibrotic tissue (FIG. 6A). Fibrotic area was quantified and expressed relative to the total tissue area (FIG. 6B). Ventricular fibrosis was prominent in mice 21 days after TAC, but not in sham-operated mice (FIGS. 6A and 6B). Strikingly, this fibrotic response was eliminated by 8MI at the highest dosage and significantly reduced in a dose-dependent manner (FIGS. 6A and 6B).

### Example 6

### 8MI is Well Tolerated In Vivo

Prior to sacrifice, serum was extracted from the blood of the mice treated as described in Example 4. Analysis of the serum chemistries revealed that significant renal and/or hepatic dysfunction was induced by TAC in 2 out of 3 control mice (#4 and #6), but only 1 out of 9 mice receiving any dose of 8MI (#14) (Table I). These results show that 8MI was well tolerated.

TABLE 2
Serum Chemistries in mice subjected to TAC or a sham
operation and treated with 8MI

| | | | | | | | Total | |
|---|---|---|---|---|---|---|---|---|
| | Treatment | | Glucose | BUN | Creatinine | Calcium | Protein | ALT |
| ID # | [8MI] | Procedure | (mg/dL) | (mg/dL) | (mg/dL) | (mg/dL) | (g/dL) | (U/L) |
| 1 | DMSO | Sham | 24 | 30 | 0.5 | 9.7 | 5.5 | 69 |
| 2 | DMSO | Sham | 135 | 26 | 0.4 | 10.7 | 6.6 | 65 |
| 3 | DMSO | Sham | 295 | 21 | 0.5 | 10.4 | 5.8 | 98 |
| 4 | DMSO | TAC | <10 | 314 | 7.5 | 11.3 | 6.1 | 536 |
| 5 | DMSO | TAC | 205 | 21 | 0.3 | 11.5 | 6.0 | 73 |
| 6 | DMSO | TAC | 188 | 27 | 0.4 | 12.2 | 6.3 | 377 |
| 7 | 5 mg/kg | TAC | 375 | 26 | 0.2 | 11.7 | 5.6 | 47 |
| 8 | 5 mg/kg | TAC | 158 | 21 | 0.2 | 9.7 | 5.8 | 53 |
| 9 | 5 mg/kg | TAC | 294 | 25 | 0.2 | 11 | 5.4 | 58 |
| 10 | 20 mg/kg | TAC | 224 | 21 | 0.2 | 9.9 | 5.3 | 51 |
| 11 | 20 mg/kg | TAC | 177 | 19 | 0.2 | 10.3 | 5.6 | 73 |
| 12 | 20 mg/kg | TAC | 326 | 28 | 0.2 | 11.1 | 6.0 | 57 |
| 13 | 40 mg/kg | TAC | 455 | 21 | 0.3 | 10.6 | 6.2 | 56 |
| 14 | 40 mg/kg | TAC | 140 | 26 | 0.6 | 8.7 | 5.5 | 308 |
| 15 | 40 mg/kg | TAC | 252 | 23 | 0.3 | 10.1 | 5.7 | 60 |

As discussed above in Example 2, 8MI showed no apparent toxicity up to 20 μM in the culture of a variety of cells. Also, mice treated with 8MI (100 mg/Kg) daily for four weeks, as described in Example 2, showed no sign of kidney or liver damage or other adverse effects. Additionally, no mortality was seen in either group (sham-operated or TAC-operated), supporting the assertion that 8MI was more tolerated in the animal model than the more potent pan-HDAC inhibitors.

### Example 7

### 8MI Blunts Transcription Associated with Cardiac Remodeling

In parallel samples from the experiment described in Example 4, the expression of a group of cardiac structural and hypertrophy-associated genes were quantified. Total RNA was extracted from left ventricular tissue using TRIzol Reagent (Invitrogen, Carlsbad, Calif.). cDNA was amplified using TaqMan Universal PCR master mix reagent (Applied Biosystems, Foster City, Calif.) under the following conditions: 2 min at 50° C., 10 min at 95° C., 40 cycles: 15 s at 95° C. and 1 min at 60° C. in an ABI 7900HT thermocycler. mRNA expression levels were normalized to those of the internal reference 18S rRNA. All samples were run in duplicates. The following primer sets were used: ANP, BNP, SERCA, MHC, and 18S. Data was analyzed using software RQ manager 1.2 from Applied Biosystems.

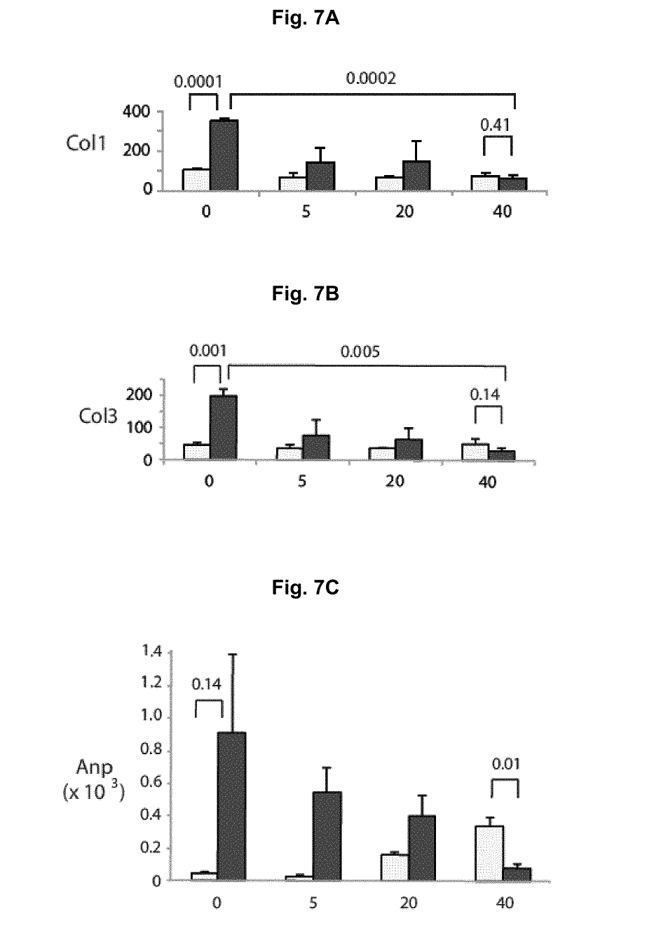

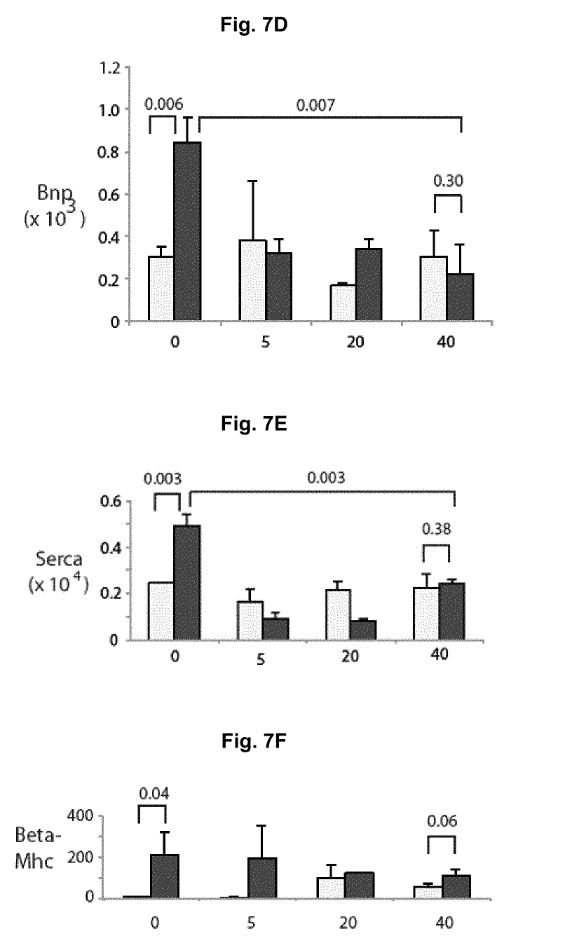

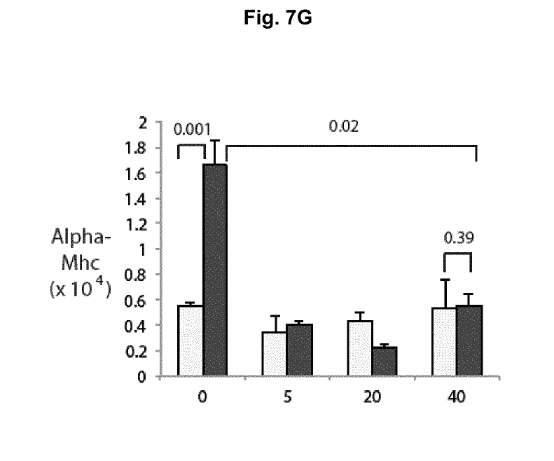

Consistent with the morphological data disclosed in Examples 4 and 5, stress-induced expression of Collagen Type 1 and Collagen Type 3 (Col1 and Col3) (FIGS. 7A and 7B), atrial natriuretic peptide (ANP) (FIG. 7C), B-type natriuretic peptide (BNP) (FIG. 7D), SERCA2 (FIG. 7E), and alpha- and beta-myosin heavy chains (FIGS. 7F and 7G) were markedly attenuated by 8MI treatment.

### Example 8

### 8MI Preserves Cardiac Function in Mice after TAC Operation

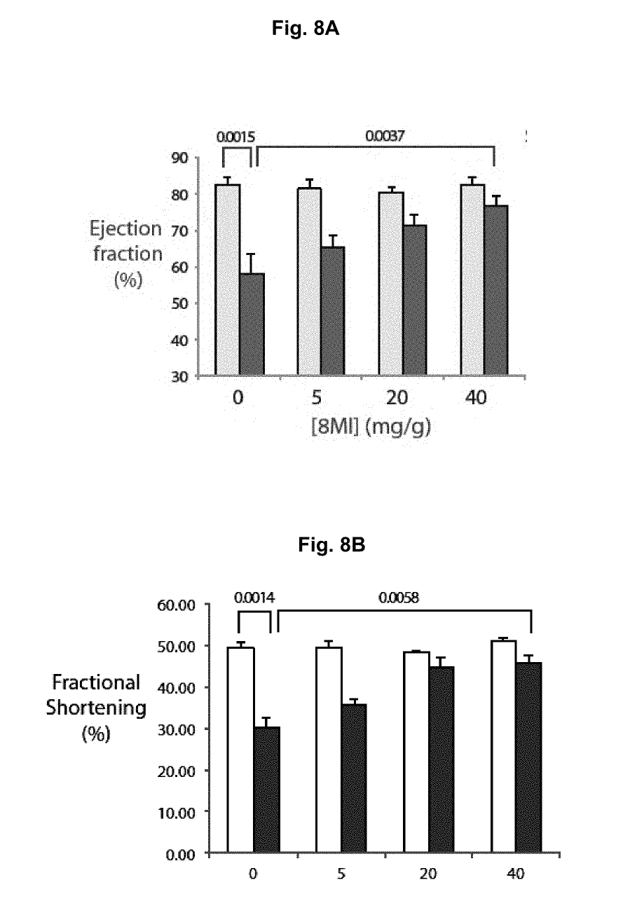

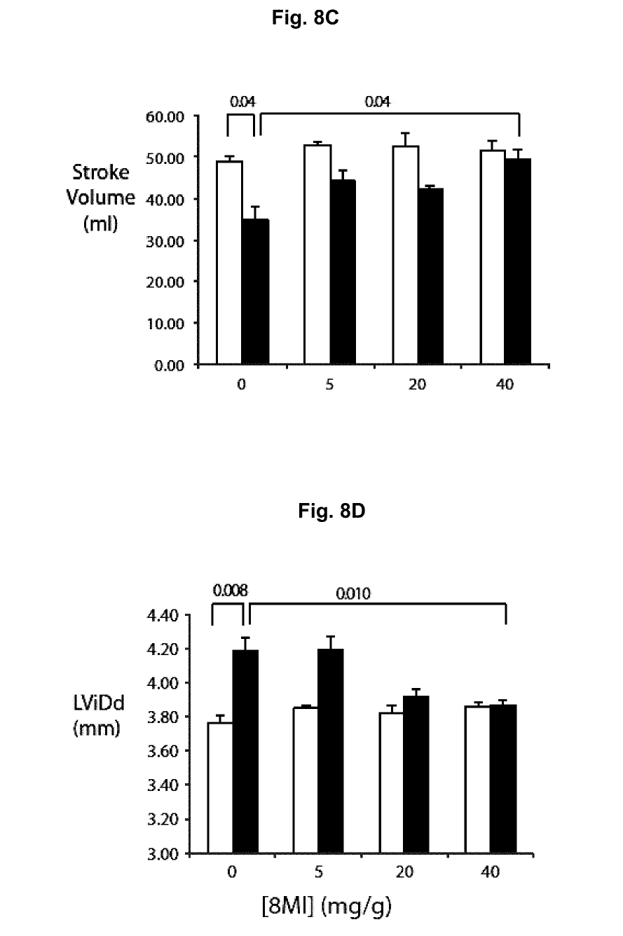

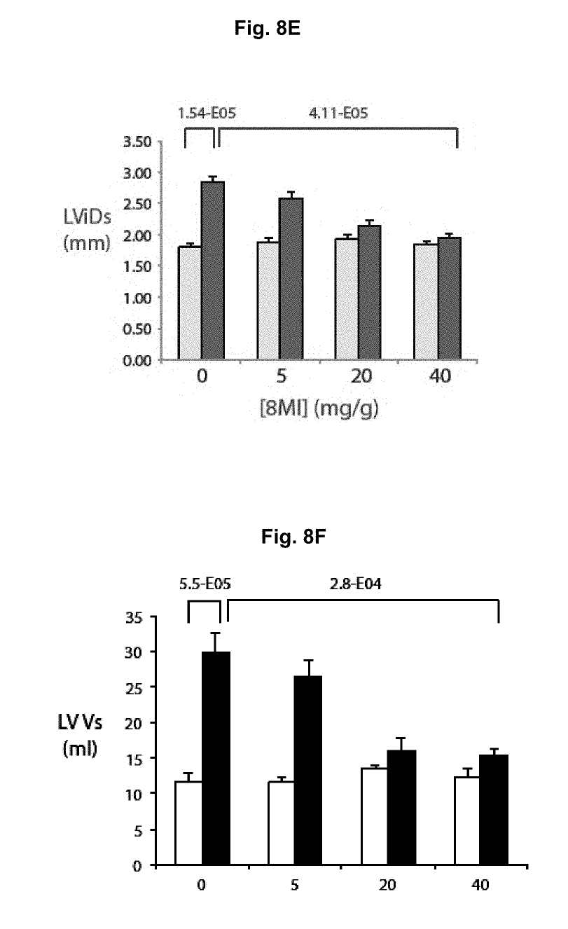

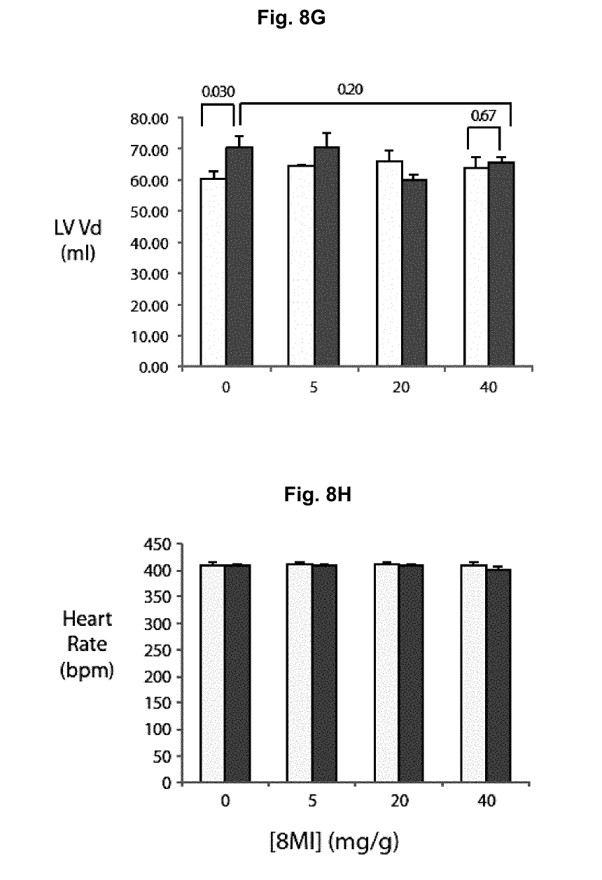

Echocardiographic studies were performed on the mice to examine systolic function under the experimental conditions described in Example 4. Echocardiographic studies revealed normal cardiac function in all mice at baseline, and in sham-operated mice treated with either DMSO or 8MI (FIGS. 8A-8H). As expected, TAC induced a 37% fall in ejection fraction at 21 days (sham, 82.4±1.8% vs. TAC, 51.7±5.1%) (FIG. 8A). Treatment with 8MI preserved systolic function despite sustained pressure overload, in a dose-dependent manner. The mice maintained a near-normal ejection fraction of 75.4% at the highest dose of 8MI (FIG. 8A). Fractional shortening (FS) (FIG. 8B) and stroke volume (FIG. 8C) were depressed by TAC and were similarly restored in the presence of 8MI. LV end diastolic diameter (LViDd) (FIG. 8D), LV end-systolic diameter (LViDs) (FIG. 8E), LV systolic volume (LV Vs) (FIG. 8F), and LV diastolic volume (LV Vd) (FIG. 8G) were increased by TAC and diminished in the presence of 8MI. These results were consistent with reduced cardiac remodeling as noted in FIG. 4B. Heart rate was unaffected in all mice (FIG. 8H).

### Example 9

### 8MI Inhibits Pressure Overload-Associated MEF2 Acetylation In Vivo

MEF2 acetylation was determined in myocardial tissue from sham- and TAC-operated mice in the presence of vehicle or 8MI, under the experimental conditions described in Example 4. Protein samples were collected in RIPA (Sigma). 500 ng of protein samples were incubated with 5 μg of acetyl lysine or GATA4 antibodies (Upstate, Charlottesville, Va.) or MEF2 antibody (Santa Cruz Biotechnology, Santa Cruz, Calif.) (5 μg). The immune-complexes were captured using TrueBlot sepharose beads and subjected to Western analysis. The immune complexes were resolved on SDS-PAGE and transferred to nitrocellulose membranes. Membranes were blocked with 5% milk in 0.5% TBS-T for 1 hour at room temperature followed by incubation in primary antibody at appropriate dilutions overnight. The membranes were incubated in HRP-conjugated secondary antibody for 2 hours at room temperature and developed using chemilum inesce.

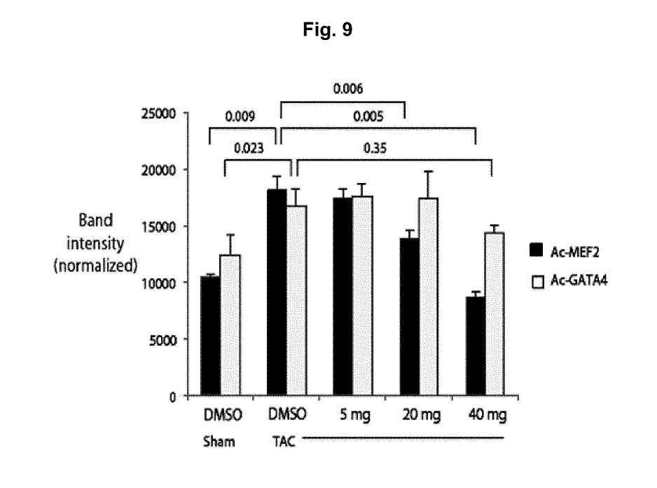

Total acetyl-MEF2 content was not different in sham-operated mice receiving the maximum dose (40 mg/kg) of 8MI versus those receiving vehicle (DMSO), suggesting a lack of effect on basal MEF2 acetylation (FIG. 9). However, TAC induced a significant increase in acetyl-MEF2 content. Increasing doses of 8MI as indicated in FIG. 9 reduced the acetyl-MEF2 content to level at or below those of the sham-operated mice. Under the same conditions, total and Ac-GATA4 levels were also increased, but no significant change was seen with 8MI.

### Example 10

### 8MI Prevents Myocyte Apoptosis During TAC

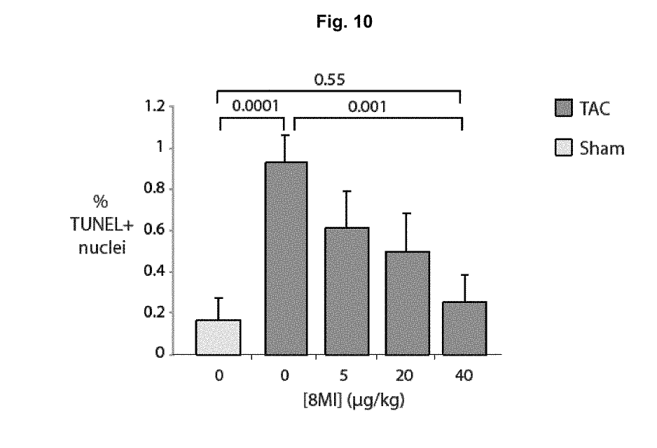

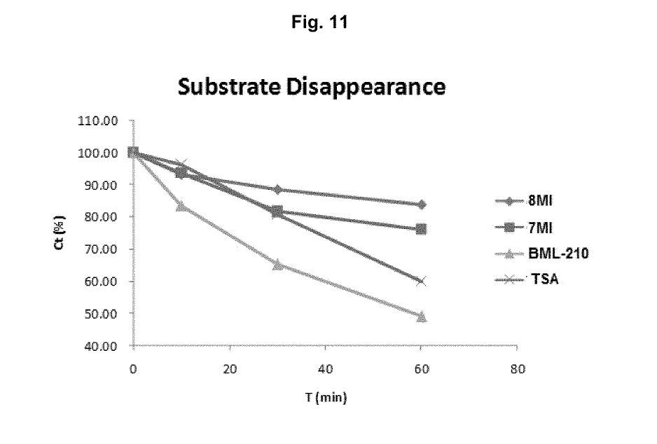

In parallel samples from the experiment described in Example 4, apoptosis was quantitated in myocardial tissue from sham- and TAC-operated mice in the presence of vehicle or 8MI. Apoptosis was detected following recommended protocol for the commercially available terminal deoxynucleotidyl transferase-mediated dUTP nick-end labeling (TUNEL) assay kit (Cardiotacs, Trevigne, Gaithersburg, Md.). Apoptosis is significantly enhanced in TAC samples in comparison to the sham samples (FIG. 10). Apoptosis was reduced in TAC-operated mice by 8MI in a dose-dependent manner (FIG. 10). At the highest dose (40 mg/kg) of 8MI, apoptosis is reduced to approximately the same level of apoptosis observed for the sham-operated mice (FIG. 10).

### Example 11

### 7MI and 8MI are Significantly more Metabolically Stable in the Liver than BML-210 or TSA

Drug clearance is a measure of the ability of the body or an organ to eliminate a drug from the blood circulation. Systemic clearance is a measure of the ability of the entire body to eliminate the drug. Organ clearance is a measure of the ability of a particular organ (hepatic or renal) to eliminate the drug. For hepatic clearance, the liver is the major organ for drug metabolism and the key organ for drug clearance. Human liver microsomes (HLM) fortified with NADPH is a standard way to measure in vitro metabolism and predict in vivo clearance. CLᵢₙₜ (intrinsic clearance) is the link between in vitro and in vivo studies, which can be estimated based on the mono-exponential decay model: Cₜ=C X e^{-kt}.

Human liver microsomes fortified with NADPH is a standard approach to evaluate metabolic stability mediated by CYP (cytochrome P450) enzymes in vitro. The reaction mixture (0.4 mL) contained 0.5 mg/mL human liver microsomes, 100 mM phosphate buffer (pH 7.4) and 5 mM of test compounds. The mixture was first warmed up for 5 min in a 37° C. shaking water bath and then NADPH at a final concentration of 1 mM was added to initiate the reaction. Aliquots (50 mL) were taken at specified time points and mixed with ice-cold methanol (containing internal standard) to stop the reaction. The mixture was vortexed briefly and centrifuged for protein precipitation. An aliquot of 10 mL supernatant was subject to LC-MS/MS analysis. The percentage of compound disappearance was used to calculate the rate of metabolism.

TABLE 3
Substrate disappearance of 7MI or 8MI from liver in
comparison to BML-210 and TSA.

| | 7MI | 8MI | BML-210 | TSA |
|---|---|---|---|---|
| K (min⁻¹) | 0.0027 | 0.0046 | 0.0116 | 0.0088 |
| Clᵢₙₜ (ml/min/kg) | 2.78 | 4.73 | 11.92 | 9.05 |
| Clₕₚ (ml/min/kg) | 2.45 | 3.85 | 7.57 | 6.30 |

K = rate constant
Clᵢₙₜ = intrinsic clearance
Clₕₚ = hepatic clearance

The results provide an indication of how a given compound would be subject to liver metabolic clearance. Although BML-210 shares structural similarity with 7MI and 8MI, the current data showed that it would be metabolically cleared much faster than 7MI or 8MI, suggesting that BML-210 is a high clearance compound (FIG. 11). Table 3 shows that BML-210 has an intrinsic clearance (Clᵢₙₜ) rate that is 4.3-fold higher than 7MI and 2.5-fold higher than 8MI. Similarly, TSA was metabolically cleared much faster than 7MI or 8MI (FIG. 11) having a Clᵢₙₜ rate that is 3.2-fold higher than 7MI and 1.9-fold higher than 8MI (Table 3). Compounds that are not metabolically stable in the liver typically display low oral bioavailability and are prone to CYP-mediated drug-drug interactions. These results show that 7MI and 8MI likely display higher oral bioavailability than BML-210 or TSA and are likely less prone to CYP-mediated drug-drug interactions.

### REFERENCES

The references, patents and published patent applications listed below, and all references cited in the specification above are hereby incorporated by reference in their entireties, as if fully set forth herein.
1. Berry, J. M., Cao, D. J., Rothermel, B. A., and Hill, J. A. (2008) Histone deacetylase inhibition in the treatment of heart disease. Expert Opin. Drug Saf. 7(1): 53-67.
2. Bisping E., Wakula, P., Poteser, M., and Heinzel, F. R. (2014) Targeting Cardiac Hypertrophy: Toward a Causal Heart Failure Therapy. J. Cardiovasc. Pharmacol. 64:293-305.
3. Cheng S, Xanthakis V, Sullivan L M, Lieb W, Massaro J, Aragam J, Benjamin E J, Vasan R S (2010) Correlates of echocardiographic indices of cardiac remodeling over the adult life course: longitudinal observations from the Framingham Heart Study. Circulation 122(6): 570-578.
4. Cohn J N, Ferrari R, Sharpe N (2000) Cardiac remodeling--concepts and clinical implications: a consensus paper from an international forum on cardiac remodeling. J Am Coll Cardiol 35(3): 569-582.
5. Cox D M, Du M, Marback M, Yang E C, Chan J, Siu K W, McDermott J C (2003) Phosphorylation motifs regulating the stability and function of myocyte enhancer factor 2A. The Journal of biological chemistry 278(17): 15297-15303.
6. Dorn G W, 2nd, Robbins J, Sugden P H (2003) Phenotyping hypertrophy: eschew obfuscation. Circ Res 92(11): 1171-1175.
7. Heidenreich, P. A., et. al., (2011) Forcasting the future of cardiovascular disease in the United States: a policy statement from the American Heart Association. Circulation (123)(8): 933-944.
8. Jayathiliaka, N., Han, A., Gaffney, K. J., Dey, R., Jarusiewicz, J. A., Noridomi, K., Philips, M. A., Lei, X., He, J., Ye, J., Gao, T., Petasis, N. A., and Chen, L. (2012) Inhibition of the function of class IIa HDACs by blocking their interaction with MEF2. Nucleic Acids Research 40(12):5378-5388.
9. Kane G C, Karon B L, Mahoney D W, Redfield M M, Roger V L, Burnett J C, Jr., Jacobsen S J, Rodeheffer R J (2011) Progression of left ventricular diastolic dysfunction and risk of heart failure. JAMA 306(8): 856-863.
10.1 Kannel W B (2000) Incidence and epidemiology of heart failure. Heart Fail Rev 5(2): 167-173.
11. Kong Y., Tannous, P., Lu, G., Berenji, K. Rothermel, B. A., Olson, E. N., and Hill, J. A. (2006) Suppressio of Class I and II Histone Deacetylases Blunts Pressure-Overload Cardiac Hypertrophy. Circulation 113: 2579-2588.
12. Lam C S, Donal E, Kraigher-Krainer E, Vasan R S (2011) Epidemiology and clinical course of heart failure with preserved ejection fraction. Eur J Heart Fail 13(1): 18-28.
13. Maron B J, Bonow R O, Cannon R O, 3rd, Leon M B, Epstein S E. (1987) Hypertrophic cardiomyopathy. Interrelations of clinical manifestations, pathophysiology, and therapy. N Engl J Med. Mar 26;316(13):780-789.
14. McKinsey, T. A. (2011) The Biology and Therapeutic Implications of HDACs in the Heart. Handbook of Experimental Pharmacology, 206: 57-77.
15. McKinsey, T. A. (2012) Therapeutic Potential for HDAC Inhibitors in the Heart. Annu. Rev. Pharmacol. Toxicol. 52: 303-19.
16. Physician's Desk Reference, 41st Ed., Publisher Edward R. Barnhart, N.J. (1987).
17. PCT/US93/0082948.
18. Remington: The Science and Practice of Pharmacy, 21st Edition, Univ. of Sciences in Philadelphia (USIP), Lippincott Williams & Wilkins, Philadelphia, Pa., 2005.
19. Samuel J L, Swynghedauw B (2008) Is cardiac hypertrophy a required compensatory mechanism in pressure-overloaded heart? J Hypertens 26(5): 857-858.
20. Spirito P, Chiarella F, Carratino L, Berisso M Z, Bellotti P, Vecchio C. (1989) Journal N Engl J Med. Clinical course and prognosis of hypertrophic cardiomyopathy in an outpatient population. 23;320(12):749-55.
21. Wei J Q, Shehadeh L A, Mitrani J M, Pessanha M, Slepak T I, Webster K A, Bishopric N H (2008) Quantitative control of adaptive cardiac hypertrophy by acetyltransferase p300. Circulation 118(9): 934-946.

1. A method of improving cardiac function in a subject comprising administering to the subject a therapeutically effective amount of one or more compounds that are independently selected from the group of compounds having a formula of 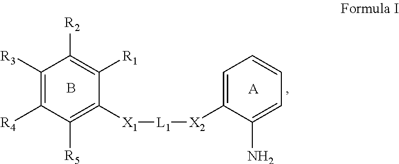
including pharmaceutically acceptable solvates, pharmaceutically acceptable prodrugs, pharmaceutically acceptable salts and pharmaceutically acceptable stereoisomers thereof, and further including mixtures thereof in all ratios, wherein:
A and B rings are independently selected from the group consisting of phenyl and pyridyl rings;
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

2. The method of claim 1, wherein the one or more compounds are independently selected from the group of compounds having a formula of: 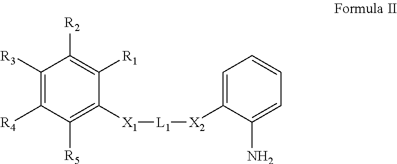
including pharmaceutically acceptable solvates, pharmaceutically acceptable prodrugs, pharmaceutically acceptable salts and pharmaceutically acceptable stereoisomers thereof, and further including mixtures thereof in all ratios, wherein:
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

3. The method of claim 2, wherein R₁-R₃, and R₅ are hydrogen and R₄ is a halogen.

4. The method of claim 2, wherein R₁, R₂, R₄, and R₅ are hydrogen and R₃ is a halogen.

5. The method of claim 2, wherein the one or more compounds have a formula of 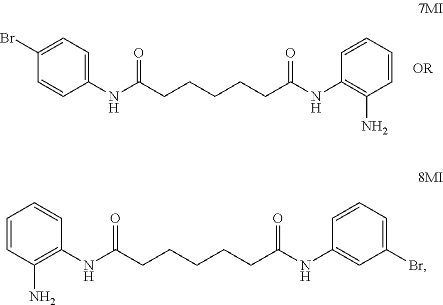 [SMILES: Nc1ccccc1NC(=O)CCCCCC(=O)Nc1ccc(Br)cc1 ; Nc1ccccc1NC(=O)CCCCCC(=O)Nc1cccc(Br)c1]
including pharmaceutically acceptable solvates, pharmaceutically acceptable prodrugs, pharmaceutically acceptable salts and pharmaceutically acceptable stereoisomers thereof, and further including mixtures thereof in all ratios.

6. The method of claim 1, wherein the one or more compounds inhibit MEF2 acetylation.

7. The method of claim 1, wherein the one or more compounds have an IC₅₀ greater than 50 μM for HDAC6 inhibition.

8. The method of claim 1, wherein the one or more compounds preferentially inhibits HDAC3 over HDAC1.

9. The method of claim 1, wherein the subject has one or more symptoms independently selected from the group consisting of: diminished diastolic function of the left ventricle, diminished systolic function of the left ventricle, diminished cardiac contractility, diminished stroke volume, diminished fractional shortening, diminished ejection fraction, increased left ventricular (LV) diastolic diameter, increased left ventricular systolic diameter, increased LV end diastolic pressure, increased ventricular wall stress, increased ventricular wall tension, increased LV systolic volume, increased LV diastolic volume, increased ventricular mass, and increased thickness of the posterior wall of the heart.

10. The method of claim 1, wherein the subject was diagnosed with one or more conditions independently selected from the group of conditions consisting of: cardiac fibrosis, hypertension, aortic stenosis, myocardial infarction, myocarditis, cardiomyopathy, valvular regurgitation, valvular disease, left ventricular dysfunction, cardiac ischemia, diastolic dysfunction, chronic angina, tachycardia, and bradycardia.

11. The method of claim 9, wherein cardiac function is improved by improving one or more of the subject's symptoms of claim 9.

12. A method of treating cardiac remodeling in a subject comprising administering to the subject a therapeutically effective amount of one or more compounds independently selected from the group of compounds having a formula of 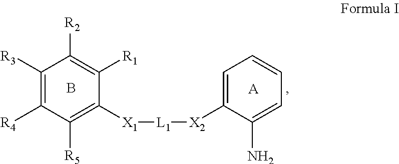
including pharmaceutically acceptable solvates, pharmaceutically acceptable prodrugs, pharmaceutically acceptable salts and pharmaceutically acceptable stereoisomers thereof, and further including mixtures thereof in all ratios, wherein:
A and B rings are independently selected from the group consisting of phenyl and pyridyl rings;
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.

13. The method of claim 12, wherein R₁-R₃, and R₅ are hydrogen and R₄ is a halogen.

14. The method of claim 12, wherein R₁, R₂, R₄, and R₅ are hydrogen and R₃ is a halogen.

15. The method of claim 12, wherein the one or more compounds have a formula of 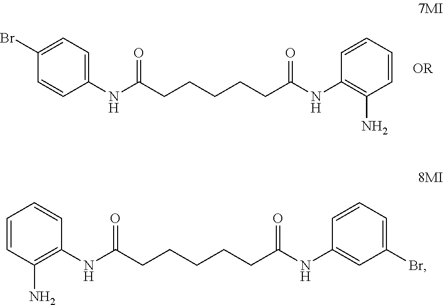 [SMILES: Nc1ccccc1NC(=O)CCCCCC(=O)Nc1ccc(Br)cc1 ; Nc1ccccc1NC(=O)CCCCCC(=O)Nc1cccc(Br)c1]
including pharmaceutically acceptable solvates, pharmaceutically acceptable prodrugs, pharmaceutically acceptable salts and pharmaceutically acceptable stereoisomers thereof, and further including mixtures thereof in all ratios.

16. The method of claim 12, wherein the cardiac remodeling is manifested as one or more symptoms selected from the group consisting of: diminished cardiac contractility, increased thickness of the posterior wall of the heart, increased ventricular mass, cardiac fibrosis, myocyte hypertrophy, myocyte necrosis, myocyte apoptosis, increased fibroblast proliferation, and increased fibrillar collagen.

17. The method of claim 12, wherein the subject was diagnosed with one or more conditions independently selected from the group consisting of: cardiac fibrosis, hypertension, aortic stenosis, myocardial infarction, myocarditis, cardiomyopathy, valvular regurgitation, valvular disease, left ventricular dysfunction, cardiac ischemia, diastolic dysfunction, chronic angina, tachycardia, and bradycardia.

18. The method of claim 12, wherein the one or more compounds inhibit MEF2 acetylation.

19. (canceled)

20. A method of treating left ventricular dysfunction in a subject comprising administering to the subject a pharmaceutically effective amount of one or more compounds are independently selected from the group of compounds having a formula of 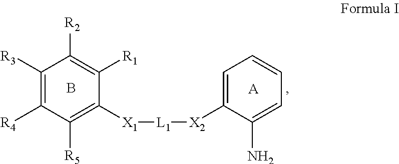
including pharmaceutically acceptable solvates, pharmaceutically acceptable prodrugs, pharmaceutically acceptable salts and pharmaceutically acceptable stereoisomers thereof, and further including mixtures thereof in all ratios, wherein:
A and B rings are independently selected from the group consisting of phenyl and pyridyl rings;
R₁-R₅ are each independently selected from the group consisting of hydrogen and halogen;
X₁ and X₂ are each independently selected from —NHC(═O)— or —C(═O)—NH—; and
L₁ is —(CH₂)ₙ—, wherein n is 4, 5, or 6.
